SEQUENCE LISTING (15 sequences)

SEQ ID NO: 1 - protein, 441 aa, Pisum sativum
MALTQLNSTCSKPQLHSSSQLSFLSRTRTRTLPRHYHSTFAPLHRTQHARISCSVAPNQVQVPAAQTQDPKGKPDCYGVFCLTYDLKAEEETKSWKKLINIAVSGAAGMISNHLLFKLASGEVFGPDQPIALKLLGSERSIQALEGVAMELEDSLFPLLREVVISIDPYEVFQDAEWALLIGAKPRGPGVERAALLDINGQIFAEQGKALNAVASRNAKVIVVGNPCNTNALICLKNAPNIPAKNFHALTRLDENRAKCQLALKAGVFYDKVSNMTIWGNHSTTQVPDFLNARIDGLPVKEVIKDNKWLEEEFTEKVQKRGGVLIQKWGRSSAASTSVSIVDAIRSLITPTPEGDWFSSGVYTNGNPYGIAEDIVFSMPCRSKGDGDYELVNDVIFDDYLRQKLAKTEAELLAEKKCVAHLTGEGIAVCDLPGDTMLPGEM

SEQ ID NO: 2 - protein, 389 aa, Pisum sativum
MSVAPNQVQVPAAQTQDPKGKPDAYGVFALTYDLKAEEETKSWKKLINIAVSGAAGMISNHLLFKLASGEVFGPDQPIALKLLGSERSIQALEGVAMELEDSLFPLLREVVISIDPYEVFQDAEWALLIGAKPRGPGVERAALLDINGQIFAEQGKALNAVASRNAKVIVVGNPCNTNALICLKNAPNIPAKNFHALTRLDENRAKAQLALKAGVFYDKVSNMTIWGNHSTTQVPDFLNARIDGLPVKEVIKDNKWLEEEFTEKVQKRGGVLIQKWGRSSAASTSVSIVDAIRSLITPTPEGDWFSSGVYTNGNPYGIAEDIVFSMPCRSKGDGDYELVNDVIFDDYLRQKLAKTEAELLAEKKCVAHLTGEGIAVADLPGDTMLPGEM

SEQ ID NO: 3 - protein, 995 aa, Synechococcus elongatus
MNQVMHPPSAEAELLSTSQSLLRQRLTLVEDIWQAVLQKECGQKLVERLNHLRATRTADGQSLNFSPSSISELIETLDLEDAIRAARAFALYFQLINSVEQHYEQREQQQFRRNLASANASEANGNSVHTEIAPTQAGTFDWLFPHLKHQNMPPQTIQRLLNQLDIRLVFTAHPTEIVRHTIRNKQRRIAGILRQLDQTEEGLKSLGTSDSWEIENIQQQLTEEIRLWWRTDELHQFKPQVLDEVDYALHYFEEVLFDTLPELSVRLQQALKASFPTLKVPTTNFCNFGSWVGGDRDGNPSVTPDVTWKTACYQRGLVLERYIASVESLSDVLSLSLHWSNVLPDLLDSLEQDQNIFPDIYETLAIRYRQEPYRLKLAYIKRRLENTLERNRRLANMPAWENKVEAADDKVYICGQEFLADLKLIRESLVQTEINCAALDKLICQVEIFSFVLTRLDFRQESTRHSDAIAEIVDYLGVLPKSYNDLSDAEKTTWLVQELKTRRPLIPKEMHFSERTVETIQTLQVLRRLQQEFGIGICQTYIISMTNEVSDVLEVLLLAQEAGLYDPLTGMTTIRIAPLFETVDDLRNAPEIMQALFEIPLYRACLAGGYEPPADGRCDETFGDRLVPNLQEIMLGYSDSNKDSGFLSSNWEIHKAQKNLQQVADPYGIDLRIFHGRGGSVGRGGGPAYAAILAQPPNTINGRIKITEQGEVLASKYSLPDLALYHLESVSTAVIQSSLLASGFDDIQPWNRIMEDLSQRSRAAYRALIYEEPDFLDFFMSVTPIPEISQLQISSRPARRKKGNKDLSSLRAIPWVFSWTQSRFLVPAWYGVGTALQGFFEEDPVENLKLMRYFYSKWPFFRMVISKVEMTLSKVDLQMASHYVHELAEKEDIPRFEKLLEQISQEYNLTKRLILEITENEALLDGDRPLQRSVQLRNGTIVPLGFLQVSLLKRLRQYTRETQASIVHFRYSKEELLRGALLTINGIAAGMRNTG

SEQ ID NO: 4 - protein, 960 aa, Sorghum bicolor
MASERHHDIDAQLRALAPGKVSEELIQYDALLVDRFLDILQDLHGPSLREFVQECYEVSADYEGKKDTSKLGELGAKLTGLAPADAILVASSILHMLNLANLAEEVELAHRRRNSKLKHGDFSDEGSATTESDIEETLKRLVSLGKTPAEVFEALKNQSVDLVFTAHPTQSARRSLLQKNARIRNCLTQLSAKDVTVEDKKELDEALHREIQAAFRTDEIRRAQPTPQDEMRYGMSYIHETVWNGVPKFLRRVDTALKNIGINERLPYDVPLIKFCSWMGGDRDGNPRVTPEVTRDVCLLSRMMAANLYINQVEDLMFELSMWRCNDELRARAEEVQSTPASKKVTKYYIEFWKQIPPNEPYRVILGAVRDKLYNTRERARHLLATGFSEISEDAVFTKIEEFLEPLELCYKSLCECGDKAIADGSLLDLLRQVFTFGLSLVKLDIRQESERQTDVIDAITTHLGIGSYRSWPEDKRMEWLVSELKGKRPLLPPDLPMTEEIADVIGAMRVLAELPIDSFGPYIISMCTAPSDVLAVELLQRECGIRQTLPVVPLFERLADLQAAPASVEKLFSTDWYINHINGKQQVMVGYSDSGKDAGRLSAAWQLYVAQEEMAKVAKKYGVKLTLFHGRGGTVGRGGGPTHLAILSQPPDTINGSIRVTVQGEVIEFMFGEENLCFQSLQRFTAATLEHGMHPPVSPKPEWRKLMEEMAVVATEEYRSVVVKEPRFVEYFRSATPETEYGKMNIGSRPAKRRPGGGITTLRAIPWIFSWTQTRFHLPVWLGVGAAFKWAIDKDIKNFQKLKEMYNEWPFFRVTLDLLEMVFAKGDPGIAGLYDELLVAEELKPFGKQLRDKYVETQQLLLQIAGHKDILEGDPYLKQGLRLRNPYITTLNVFQAYTLKRIRDPSFKVTPQPPLSKEFADENKPAGLVKLNGERVPPGLEDTLILTMKGIAAGMQNTG

SEQ ID NO: 5 - protein, 463 aa, Synechococcus elongatus
MVALTPNPSYSVTIQLRIPNRAGTLANVTQAIANVGGSLGNIELVERDLKFLIRNITVDASSEKHAGDIVDAVKAVPEIEILKVLDRTFAIHQGGKITVESRIPLTVQSDLAMAYTPGVGRVCQAIAEKPEQVYDLTVKSNMVAIVTDGSAVLGLGNLGPEAAMPVMEGKAMLFKEFAGVNAFPICLATQDVEEIVRTVKYLAPVFGGVNLEDIAAPRCFEIEQRLKAETDIPIFHDDQHGTAIVSLAALFNALKLVKKDLQSVKIVINGAGAAGVAIAKLLRQAGATDIWMCDSKGIISSQRDNLTPEKRAFAVAASGTLAEAMQDADIFLGVSAPGVVTPAMVTSMAKDPIVFAMANPIPEIQPELIMDKVAVVATGRSDYPNQINNVLAFPGVFRGALDCRASSITTNMCLQAAQAIASLVSPAQLDREHIIPSVFDQRVAAAVAAAVGQAARQDGVARR

SEQ ID NO: 6 - protein, 268 aa, Corynebacterium glutamicum
MRFGRIATPDGMCFCSIEGEGDDVANLTAREIEGTPFTEPKFTGREWPLKDVRLLAPMLPSKVVAIGRNYADHVAEVFKKSAESLPPTLFLKPPTAVTGPESPIRIPSFATKVEFEGELAVVIGKPCKNVKADDWKSVVLGFTIINDVSSRDLQFADGQWARAKGIDTFGPIGPWIETDINSIDLDNLPIKARLTHDGETQLKQDSNSNQMIMKMGEIIEFITASMTLLPGDVIATGSPAGTEAMVDGDYIEIEIPGIGKLGNPVVDA

SEQ ID NO: 7 - protein, 540 aa, Escherichia coli
MRVNNGLTPQELEAYGISDVHDIVYNPSYDLLYQEELDPSLTGYERGVLTNLGAVAVDTGIFTGRSPKDKYIVRDDTTRDTFWWADKGKGKNDNKPLSPETWQHLKGLVTRQLSGKRLFVVDAFCGANPDTRLSVRFITEVAWQAHFVKNMFIRPSDEELAGFKPDFIVMNGAKCTNPQWKEQGLNSENFVAFNLTERMQLIGGTWYGGEMKKGMFSMMNYLLPLKGIASMHCSANVGEKGDVAVFFGLSGTGKTTLSTDPKRRLIGDDEHGWDDDGVFNFEGGCYAKTIKLSKEAEPEIYNAIRRDALLENVTVREDGTIDFDDGSKTENTRVSYPIYHIDNIVKPVSKAGHATKVIFLTADAFGVLPPVSRLTADQTQYHFLSGFTAKLAGTERGITEPTPTFSACFGAAFLSLHPTQYAEVLVKRMQAAGAQAYLVNTGWNGTGKRISIKDTRAIIDAILNGSLDNAETFTLPMFNLAIPTELPGVDTKILDPRNTYASPEQWQEKAETLAKLFIDNFDKYTDTPAGAALVAAGPKL

SEQ ID NO: 8 - protein, 510 aa, Escherichia coli
MRIGIPRERLTNETRVAATPKTVEQLLKLGFTVAVESGAGQLASFDDKAFVQAGAEIVEGNSVWQSEIILKVNAPLDDEIALLNPGTTLVSFIWPAQNPELMQKLAERNVTVMAMDSVPRISRAQSLDALSSMANIAGYRAIVEAAHEFGRFFTGQITAAGKVPPAKVMVIGAGVAGLAAIGAANSLGAIVRAFDTRPEVKEQVQSMGAEFLELDFKEEAGSGDGYAKVMSDAFIKAEMELFAAQAKEVDIIVTTALIPGKPAPKLITREMVDSMKAGSVIVDLAAQNGGNCEYTVPGEIFTTENGVKVIGYTDLPGRLPTQSSQLYGTNLVNLLKLLCKEKDGNITVDFDDVVIRGVTVIRAGEITWPAPPIQVSAQPQAAQKAAPEVKTEEKCTCSPWRKYALMALAIILFGWMASVAPKEFLGHFTVFALACVVGYYVVWNVSHALHTPLMSVTNAISGIIVVGALLQIGQGGWVSFLSFIAVLIASINIFGGFTVTQRMLKMFRKN

SEQ ID NO: 9 - protein, 462 aa, Escherichia coli
MSGGLVTAAYIVAAILFIFSLAGLSKHETSRQGNNFGIAGMAIALIATIFGPDTGNVGWILLAMVIGGAIGIRLAKKVEMTEMPELVAILHSFVGLAAVLVGFNSYLHHDAGMAPILVNIHLTEVFLGIFIGAVTFTGSVVAFGKLCGKISSKPLMLPNRHKMNLAALVVSFLLLIVFVRTDSVGLQVLALLIMTAIALVFGWHLVASIGGADMPVVVSMLNSYSGWAAAAAGFMLSNDLLIVTGALVGSSGAILSYIMCKAMNRSFISVIAGGFGTDGSSTGDDQEVGEHREITAEETAELLKNSHSVIITPGYGMAVAQAQYPVAEITEKLRARGINVRFGIHPVAGRLPGHMNVLLAEAKVPYDIVLEMDEINDDFADTDTVLVIGANDTVNPAAQDDPKSPIAGMPVLEVWKAQNVIVFKRSMNTGYAGVQNPLFFKENTHMLFGDAKASVDAILKAL

SEQ ID NO: 10 - protein, 1803 aa, Euglena gracilis
MKQSVRPIISNVLRKEVALYSTIIGQDKGKEPTGRTYTSGPKPASHIEVPHHVTVPATDRTPNPDAQFFQSVDGSQATSHVAYALSDTAFIYPITPSSVMGELADVWMAQGRKNAFGQVVDVREMQSEAGAAGALHGALAAGAIATTFTASQGLLLMIPNMYKIAGELMPSVIHVAARELAGHALSIFGGHADVMAVRQTGWAMLCSHTVQQSHDMALISHVATLKSSIPFVHFFDGFRTSHEVNKIKMLPYAELKKLVPPGTMEQHWARSLNPMHPTIRGTNQSADIYFQNMESANQYYTDLAEVVQETMDEVAPYIGRHYKIFEYVGAPDAEEVTVLMGSGATTVNEAVDLLVKRGKKVGAVLVHLYRPWSTKAFEKVLPKTVKRIAALDRCKEVTALGEPLYLDVSATLNLFPERQNVKVIGGRYGLGSKDFIPEHALAIYANLASENPIQRFTVGITDDVTGTSVPFVNERVDTLPEGTRQCVFWGIGSDGTVGANRSAVRIIGDNSDLMVQAYFQFDAFKSGGVTSSHLRFGPKPITAQYLVTNADYIACHFQEYVKRFDMLDAIREGGTFVLNSRWTTEDMEKEIPADFRRNVAQKKVRFYNVDARKICDSFGLGKRINMLMQACFFKLSGVLPLAEAQRLLNESIVHEYGKKGGKVVEMNQAVVNAVFAGDLPQEVQVPAAWANAVDTSTRTPTGIEFVDKIMRPLMDFKGDQLPVSVMTPGGTFPVGTTQYAKRAIAAFIPQWIPANCTQCNYCSYVCPHATIRPFVLTDQEVQLAPESFVTRKAKGDYQGMNFRIQVAPEDCTGCQVCVETCPDDALEMTDAFTATPVQRTNWEFAIKVPNRGTMTDRYSLKGSQFQQPLLEFSGACEGCGETPYVKLLTQLFGERTVIANATGCSSIWGGTAGLAPYTTNAKGQGPAWGNSLFEDNAEFGFGIAVANAQKRSRVRDCILQAVEKKVADEGLTTLLAQWLQDWNTGDKTLKYQDQIIAGLAQQRSKDPLLEQIYGMKDMLPNISQWIIGGDGWANDIGFGGLDHVLASGQNLNVLVLDTEMYSNTGGQASKSTHMASVAKFALGGKRTNKKNLTEMAMSYGNVYVATVSHGNMAQCVKAFVEAESYDGPSLIVGYAPCIEHGLRAGMARMVQESEAAIATGYWPLYRFDPRLATEGKNPFQLDSKRIKGNLQEYLDRQNRYVNLKKNNPKGADLLKSQMADNITARFNRYRRMLEGPNTKAAAPSGNHVTILYGSETGNSEGLAKELATDFERREYSVAVQALDDIDVADLENMGFVVIAVSTCGQGQFPRNSQLFWRELQRDKPEGWLKNLKYTVFGLGDSTYYFYCHTAKQIDARLAALGAQRVVPIGFGDDGDEDMFHTGFNNWIPSVWNELKTKTPEEALFTPSIAVQLTPNATPQDFHFAKSTPVLSITGAERITPADHTRNFVTIRWKTDLSYQVGDSLGVFPENTRSVVEEFLQYYGLNPKDVITIENKGSRELPHCMAVGDLFTKVLDILGKPNNRFYKTLSYFAVDKAEKERLLKIAEMGPEYSNILSEMYHYADIFHMFPSARPTLQYLIEMIPNIKPRYYSISSAPIHTPGEVHSLVLIDTWITLSGKHRTGLTCTMLEHLQAGQVVDGCIHPTAMEFPDHEKPVVMCAMGSGLAPFVAFLRERSTLRKQGKKTGNMALYFGNRYEKTEFLMKEELKGHINDGLLTLRCAFSRDDPKKKVYVQDLIKMDEKMMYDYLVVQKGSMYCCGSRSFIKPVQESLKHCFMKAGGLTAEQAENEVIDMFTTGRYNIEAW

SEQ ID NO: 11 - protein, 1945 aa, Cryptosporidium muris
MKSEIVDGCVAACHIAYACSEVAFIYPITPSSSISEAADSWMVKGKKNLFDQVVSVVEMQSEMGSAGALHGSLCVGCVTTTFTASQGLLLMIPNMYKIAGELWPCVFHVTARALATSSLSIFGDHNDIMAARQTGWAFLGAMTVQEVMDLALVAHISTLESSMPFVHFFDGFRTSHELQKIEMIDYDTIKALYPYDKLRAFRSRALNPTHPVLRGTATSSDVYFQTVESRNAYYDAVPTIVQDVMNKVAKYTGRQYNLFDYYGYKEAEYVIVVMGSGGLTIEEMIEYLIKESNEKVGMIKVRLFRPWSPDTFAKVLPTTVRRITVLERCKESGALGEPLYLDVSTTIMRIMQSDSRYKNISVIGGRYGLASKEFTPGMALSIWENMRSESPIQNFSVGINDDVTFKSLQIRQPKLDLLTDETRQCMFWGLGSDGTVSANKNAIKIIGESTNLFVQGYFAYDAKKAGGATMSHLRFGPKPIKSPYLLQRCDYIAVHHPSYIYKFDVLENIKENGIFVLNCSWKSVDKISEELPARIKSIIARNNIRMYVVDAQDVAIRANLGRRINNILMVAFFRLANIIPFEEAINLIKDAIQKSYSKKGEAVIKSNWRAVDLALESLIEVKYNRDAWLSSFSNQIVGNGYEISKGIIEEYPYSKTTSETSTCESPFSKKQIQISINEKPDLNKFVSDVLEPVNALKGDNLPVSVFDPSGVVPLGTTAFEKRGIAISIPIVDMNKCTQCNYCSIVCPHAAIRPFLLEEVEFEEAPKSMHILRAKGGAEFSSYYYRIQVAPLDCTGCELCVHACPDDALHMEPLQMVRNQEIPHWNYLVKLPNHGYKFDKSTVKGSQFQKPLLEFSAACEGCGETPYIKLLTQLFGERMVIANATGCSSIWGASFPSVPYTVTDKGYGPAWGNSLFEDNAEYGLGMVVGYRQRRTRIEALIKEFLNKSDDQKLKNIHEKSAIKDVYLKFEDYLRSWLKNMNEGDVCQYLYEKITTTIEENLECNKFDTLLSDEHLEMLRRIYQDRDLFPKISHWIVGGDGWAYDIGYAGLDHVLAYGEDVNILILDTEVYSNTGGQTSKSTPFGAVAKFSQGGNLRQKKDLGLIAMEYGSVYVASIALGANYQQTIRSLMEAERYPGTSLIIAYSTCIEHGYDKYTLQQESVKLAVESGYWPLYRFNPQLLKFDEINNTIVTLSTGFTLDSKKIKADISQFLKRENRFLQLFRSNPELASITQSRLKIYSDRRFQHMKNLSENLSVTSLKDQVKKLKDKLLALQNGEAGGGDLNLQFERNMHILYGTETGNSEDVALYIQAELTSRGYTSTVCNLDDIDIDEFLDPSQYSSFILVTSTAGQGEFPGSSKILYESLERRYIELLSNGEDVKFLCNFMQYGVFGLGDSTYVYFNEAAKKWDKLLSDCGAVRIGRMGLGDDQSDEKYETDLIEWLPDYLQLVNAPEPSNTDDQPKDPLYNVQVIENIYRNDQLNIQTGTLHAINYEGNCDIPYTPILPPNSILLPLIENTRITSLDHDRDVRHLIFDLSDDSLHKNNLRYNLGDSLALYAQNDFEEAKKACEFFGFNPYSIIEINLNQIETNKNIRVNQRYLSIFGMKMTILQLFVECLDLWGKPGRRFYHEFYRYCSGSEKEHAKKWSRNEGKSLIQEFQSETKTFIDMFYLYPSAKPSLSQLLDIVPLIKPRYYSIASSCKYVNNSKIELCVGIVDWNTSSGILKYGQCTGFINRLPKLISKESNEGIMSDTSNFDIVPVLPCSLKSSAFNLPKDNMSPIIMACMGTGLAPFRAFIQYKYYVKTVLKQEIGPVILYFGCRYKNKDYLYREELEQYVNDGIITSLNVAFSRDPIEDKKQKLCKDSRIRYRQKVYVQRIMEENSSELHENLIDKEGYFYLCGTKQVPIDIRKAIVNIIMSQDSNATEESANEILNGLQIKGRYNIEAWS

SEQ ID NO: 12 - protein, 1178 aa, Synechococcus elongatus
MATNTIATLDANEAVAKVAYKLNEVIAIYPITPASLMGEWADAWASQGQPNLWGTVPSIIEMQSEGGAAGAVHGALQTGSLTTTFTASQGLLLMIPNLYKIAGELTSAVIHVAARSVAAQALSIFGDHSDVMAVRGTGFALLSSASVQEAHDMALIAQAATMKARVPFIHFFDGFRTSHEIQKIELLDESVLRELIDDEDVFAHRARALTPDHPVVRGTAQNPDVFFQARESVNPFYDKCSAIVKEMMDRFGALTGRAYKLFEYVGAPDATRVIMLMGSGCETVHETVDYLNAQGEKVGVLKVRLYRPFDGSALISALPKTVEKIAVLDRTKEPGANGEPLYLDVVSALMEAWEGTMPKVVGGRYGLSSKEFNPAMVKGIFDELDQAKPKNHFTVGINDDVSHTSLAYDPSFSSEPDSVVRAMFYGLGSDGTVGANKNSIKIIGEETDNYAQGYFVYDSKKSGAVTVSHLRFGPNLIRSTYLINQANFVGCHQWLFLEKLDVLSGAKDGSIFLLNSPYAVDQVWDQLPLEVQEQIFHKNLKFYVINANKVARESGMGGRINTVMQTCFFALSGVLPKEEAISKIKEYIQKTYGKKGADVVTMNIQAVDNTLANLFEVNVGEANSPIRKPPAVSPNAPDFMRNVQAPMLIKEGDRLPVSCLPCDGTYPTGTSKWEKRNVAQFIPEWDPEVCIQCGKCVMVCPHATIRAKVYEPNLLGNAPESFKSIDAKDKNFSGQKFTIQVAPEDCTGCGVCVDVCPAKNKAQPSKKAINMVEQLPLREQERTNWDYFLNLPLPERRELKLNQIREQQLQEPLFEFSGACAGCGETPYIKLVSQLFGDRTVIANATGCSSIYGGNLPTTPYTTNAEGKGIAWSNSLFEDNAEFGLGFRLSIDKQAQFAAELLQRLSGELGDSFVGELLNARQADEADIWEQRQRVRELKNKLATLNSPDAKQLASLADYLVKKSVWIVGGDGWAYDIGFGGLDHAIASGKNINILVMDTEVYSNTGGQSSKATPRAAVAKFAAGGKPAPKKDLGLIAMTYGNVYVASVAMGARDEHTLKAFLEAEAYEGPSLIIAYSHCIAHGINMQTAMSHQKELVESGRWLLYRYNPDLKTEGKNPLQLDSRTPKGSVESSMYKENRFKMLTMTKPKAAKELLKQAQNDVDTRWRMYEYLANRPEA

SEQ ID NO: 13 - protein, 568 aa, Zymomonas mobilis
MSYTVGTYLAERLVQIGLKHHFAVAGDYNLVLLDNLLLNKNMEQVYCCNELNCGFSAEGYARAKGAAAAVVTYSVGALSAFDAIGGAYAENLPVILISGAPNNNDHAAGHVLHHALGKTDYHYQLEMAKNITAAAEAIYTPEEAPAKIDHVIKTALREKKPVYLEIACNIASMPCAAPGPASALFNDEASDEASLNAAVEETLKFIADRDKVAVLVGSKLRAAGAEEAAVKFADALGGAVATMAAAKSFFPEENPHYIGTSWGEVSYPGVEKTMKEADAVIALAPVFNDYSTTGWTDIPDPKKLVLAEPRSVVVNGIRFPSVHLKDYLTRLAQKVSKKTGALDFFKSLNAGELKKAAPADPSAPLVNAEIARQVEALLTPNTTVIAETGDSWFNAQRIKLPNGARVEYEMQWGHIGWSVPAAFGYAVGAPERRNILMVGDGSFQLTAQEVAQMVRLKPPVIIFLINNYGYTIEVMIHDGPYNNIKNWDYAGLMEVFNGNGGYDSGAGKGLKAKTGGELAEAIKVALANTDGPTLIECFIGREDCTEELVKWGKRVAAANSRKPVNKLL

SEQ ID NO: 14 - protein, 512 aa, Escherichia coli
MTNNPPSAQIKPGEYGFPLKLKARYDNFIGGEWVAPADGEYYQNLTPVTGQLLCEVASSGKRDIDLALDAAHKVKDKWAHTSVQDRAAILFKIADRMEQNLELLATAETWDNGKPIRETSAADVPLAIDHFRYFASCIRAQEGGISEVDSETVAYHFHEPLGVVGQIIPWNFPLLMASWKMAPALAAGNCVVLKPARLTPLSVLLLMEIVGDLLPPGVVNVVNGAGGVIGEYLATSKRIAKVAFTGSTEVGQQIMQYATQNIIPVTLELGGKSPNIFFADVMDEEDAFFDKALEGFALFAFNQGEVCTCPSRALVQESIYERFMERAIRRVESIRSGNPLDSVTQMGAQVSHGQLETILNYIDIGKKEGADVLTGGRRKLLEGELKDGYYLEPTILFGQNNMRVFQEEIFGPVLAVTTFKTMEEALELANDTQYGLGAGVWSRNGNLAYKMGRGIQAGRVWTNCYHAYPAHAAFGGYKQSGIGRETHKMMLEHYQQTKCLLVSYSDKPLGLF

SEQ ID NO: 15 - protein, 652 aa, Escherichia coli
MSQIHKHTIPANIADRCLINPQQYEAMYQQSINVPDTFWGEQGKILDWIKPYQKVKNTSFAPGNVSIKWYEDGTLNLAANCLDRHLQENGDRTAIIWEGDDASQSKHISYKELHRDVCRFANTLLELGIKKGDVVAIYMPMVPEAAVAMLACARIGAVHSVIFGGFSPEAVAGRIIDSNSRLVITSDEGVRAGRSIPLKKNVDDALKNPNVTSVEHVVVLKRTGGKIDWQEGRDLWWHDLVEQASDQHQAEEMNAEDPLFILYTSGSTGKPKGVLHTTGGYLVYAALTFKYVFDYHPGDIYWCTADVGWVTGHSYLLYGPLACGATTLMFEGVPNWPTPARMAQVVDKHQVNILYTAPTAIRALMAEGDKAIEGTDRSSLRILGSVGEPINPEAWEWYWKKIGNEKCPVVDTWWQTETGGFMITPLPGATELKAGSATRPFFGVQPALVDNEGNPLEGATEGSLVITDSWPGQARTLFGDHERFEQTYFSTFKNMYFSGDGARRDEDGYYWITGRVDDVLNVSGHRLGTAEIESALVAHPKIAEAAVVGIPHNIKGQAIYAYVTLNHGEEPSPELYAEVRNWVRKEIGPLATPDVLHWTDSLPKTRSGKIMRRILRKIAAGDTSNLGDTSTLADPGVVEKLLEEKQAIAMPS

(19) United States
(12) Patent Application Publication
(10) Pub. No.: US 2015/0203824 A1
(43) Pub. Date: Jul. 23, 2015

(54) METHODS AND COMPOSITIONS FOR THE AUGMENTATION OF PYRUVATE AND ACETYL-COA FORMATION

(71) Applicant: JOULE UNLIMITED TECHNOLOGIES, INC., Bedford, MA (US)

(72) Inventor: Frank A. Skraly, Watertown, MA (US)

(21) Appl. No.: 14/417,540

(22) PCT Filed: Jul. 26, 2013
(86) PCT No.: PCT/US2013/052346

§ 371 Date: Jan. 26, 2015

Related U.S. Application Data
(60) Provisional application No. 61/676,215, filed on Jul. 26, 2012.

(51) Int. Cl. C12N 9/04; C12N 9/02; C12N 9/88; C12P 19/40; C12P 7/40

(52) U.S. Cl. CPC C12N 9/0006; C12P 19/40; C12P 7/40; C12N 9/88; C12N 9/0008; C12Y 101/01082; C12Y 401/01031; C12Y 101/0104; C12Y 401/01003; C12Y 101/01037; C12Y 102/04001; C12Y 102/07005

(57) ABSTRACT

The present disclosure identifies methods and compositions for modifying photoautotrophic organisms as hosts, such that the organisms efficiently convert carbon dioxide and light into pyruvate or acetyl-CoA, and in particular the use of such organisms for the commercial production of molecules derived from these precursors, e.g., ethanol.

### CROSS-REFERENCE TO RELATED APPLICATIONS

This application claims the benefit of U.S. Provisional Patent Application No. 61/676,215, filed Jul. 26, 2012, the disclosure of which is incorporated herein by reference.

### SEQUENCE LISTING

The instant application contains a Sequence Listing which has been submitted in ASCII format via EFS-Web and is hereby incorporated by reference in its entirety. Said ASCII copy, created on July 26, 2013 is named 24148PCT_CRF_Sequencelisting.txt and is 101,562 bytes in size.

### FIELD OF THE INVENTION

The present disclosure relates to methods for conferring pyruvate and/or acetyl-CoA-producing properties to a heterotrophic or photoautotrophic host, such that the modified host can be used in the commercial production of carbon-based compounds of interest.

### BACKGROUND OF THE INVENTION

It has been established that pyruvate formation, especially under autotrophic conditions, originates not from pyruvate kinase (Pyk) as is often the case in heterotrophs, but rather from the NADP+-dependent malic enzyme (Nogales et al., 2012. “Detailing the optimality of photosynthesis in cyanobacteria through systems biology analysis”. Proc. Natl. Acad. Sci. USA 109(7):2678-2683; http://www.pnas.org/content/109/7/2678). What is needed therefore, are biosynthetic pathways alternative to Pyk for biosynthesis of pyruvate or acetyl-CoA.

### SUMMARY OF THE INVENTION

The present invention provides, in certain embodiments, an engineered photosynthetic microbe, wherein the engineered photosynthetic microbe comprises a recombinant MdhP enzyme. In one embodiment, the recombinant MdhP enzyme is a Pisum sativum MdhP enzyme. In another embodiment, the MdhP enzyme is at least 95% identical to SEQ ID NO: 1. In still another embodiment the MdhP enzyme refers to an enzyme with the amino acid sequence of SEQ ID NO: 1 or a homolog thereof, wherein a MdhP homolog is a protein whose BLAST alignment (i) covers >90% length of SEQ ID NO: 1, (ii) covers >90% of the length of the matching protein, and (iii) has >50% identity with SEQ ID NO: 1 (when optimally aligned using the parameters provided herein). In yet another embodiment, the MdhP enzyme is at least 95% identical to SEQ ID NO: 2. In still another embodiment the MdhP enzyme refers to an enzyme with the amino acid sequence of SEQ ID NO: 2 or a homolog thereof, wherein a MdhP homolog is a protein whose BLAST alignment (i) covers >90% length of SEQ ID NO: 2, (ii) covers >90% of the length of the matching protein, and (iii) has >50% identity with SEQ ID NO: 2 (when optimally aligned using the parameters provided herein).

In one aspect, the engineered photosynthetic microbe comprises an additional mutation which reduces the expression or activity of its endogenous Mdh enzyme. In a further aspect, the mutation is a knockout of the gene encoding the endogenous Mdh enzyme. In yet another aspect, the engineered photosynthetic microbe further comprises a recombinant phosphoenol pyruvate carboxylase. In still another aspect, the engineered photosynthetic microbe further comprises a recombinant NADPH-linked malic enzyme. In still another aspect, the engineered photosynthetic microbe further comprises a recombinant phosphoenol pyruvate carboxylase and a recombinant NADPH-linked malic enzyme.

In a related embodiment, the recombinant phosphoenol pyruvate carboxylase is the S8D mutant phosphoenol pyruvate carboxylase. In another embodiment, the S8D mutant phosphoenol pyruvate carboxylase is derived from Sorghum ppc. In still another embodiment, the recombinant phosphoenol pyruvate carboxylase is at least 95% identical to SEQ ID NO: 4. In still another embodiment the recombinant phosphoenol pyruvate carboxylase enzyme refers to an enzyme with the amino acid sequence of SEQ ID NO: 4 or a homolog thereof, wherein a recombinant phosphoenol pyruvate carboxylase homolog is a protein whose BLAST alignment (i) covers >90% length of SEQ ID NO: 4, (ii) covers >90% of the length of the matching protein, and (iii) has >50% identity with SEQ ID NO: 4 (when optimally aligned using the parameters provided herein).

In another aspect, the recombinant NADPH-linked malic enzyme is the Synechococcus elongatus PPC 7002 NADPH-linked malic enzyme. In yet another aspect, the recombinant NADPH-linked malic enzyme is at least 95% identical to SEQ ID NO: 5. In still another aspect the recombinant NADPH-linked malic enzyme refers to an enzyme with the amino acid sequence of SEQ ID NO: 5 or a homolog thereof, wherein a recombinant NADPH-linked malic enzyme homolog is a protein whose BLAST alignment (i) covers >90% length of SEQ ID NO: 5, (ii) covers >90% of the length of the matching protein, and (iii) has >50% identity with SEQ ID NO: 5 (when optimally aligned using the parameters provided herein).

The present invention further provides an engineered photosynthetic microbe, wherein the engineered photosynthetic microbe comprises a recombinant oxaloacetate decarboxylase. In one aspect, the oxaloacetate decarboxylase is Corynebacterium glutamicum oxaloacetate decarboxylase. In another aspect, the recombinant oxaloacetate decarboxylase is at least 95% identical to SEQ ID NO: 6. In still another aspect the recombinant oxaloacetate decarboxylase enzyme refers to an enzyme with the amino acid sequence of SEQ ID NO: 6 or a homolog thereof, wherein a recombinant oxaloacetate decarboxylase homolog is a protein whose BLAST alignment (i) covers >90% length of SEQ ID NO: 6, (ii) covers >90% of the length of the matching protein, and (iii) has >50% identity with SEQ ID NO: 6 (when optimally aligned using the parameters provided herein).

In one embodiment, the engineered photosynthetic microbe comprises a recombinant phosphoenol pyruvate carboxylase. In another embodiment, the recombinant phosphoenol pyruvate carboxylase is at least 95% identical to SEQ ID NO: 3. In still another embodiment, the recombinant phosphoenol pyruvate carboxylase enzyme refers to an enzyme with the amino acid sequence of SEQ ID NO: 3 or a homolog thereof, wherein a recombinant phosphoenol pyruvate carboxylase homolog is a protein whose BLAST alignment (i) covers >90% length of SEQ ID NO: 3, (ii) covers >90% of the length of the matching protein, and (iii) has >50% identity with SEQ ID NO: 3 (when optimally aligned using the parameters provided herein). In one aspect, the engineered photosynthetic microbe comprises an endogenous, non-recombinant phosphoenol pyruvate carboxylase.

In one aspect, the engineered photosynthetic microbe comprises a recombinant phosphoenolpyruvate carboxykinase. In another aspect, the recombinant phosphoenolpyruvate carboxykinase is derived from E. coli. In still another aspect, the recombinant phosphoenolpyruvate carboxykinase is at least 95% identical to SEQ ID NO: 7. In yet another aspect, the recombinant phosphoenolpyruvate carboxykinase enzyme refers to an enzyme with the amino acid sequence of SEQ ID NO: 7 or a homolog thereof, wherein a recombinant phosphoenolpyruvate carboxykinase homolog is a protein whose BLAST alignment (i) covers >90% length of SEQ ID NO: 7, (ii) covers >90% of the length of the matching protein, and (iii) has >50% identity with SEQ ID NO: 7 (when optimally aligned using the parameters provided herein).

In one embodiment, the engineered photosynthetic microbe lacks an endogenous or recombinant malate dehydrogenase activity, or comprises a mutation which attenuates or knocks out endogenous malate dehydrogenase activity in the engineered photosynthetic microbe. In another embodiment, the engineered photosynthetic microbe comprises a mutation which attenuates or knocks out endogenous pyruvate dehydrogenase activity in the photosynthetic microbe.

The present invention further provides an engineered photosynthetic microbe, wherein the engineered photosynthetic microbe comprises a recombinant NADPH-producing transhydrogenase system. In one embodiment, the recombinant NADPH-producing transhydrogenase system comprises PntA transhydrogenase, PntB transhydrogenase, and/or PntAB transhydrogenase. In a further embodiment, the PntA transhydrogenase is at least 95% identical to SEQ ID NO: 8. In yet another embodiment, the PntA transhydrogenase enzyme refers to an enzyme with the amino acid sequence of SEQ ID NO: 8 or a homolog thereof, wherein a PntA transhydrogenase homolog is a protein whose BLAST alignment (i) covers >90% length of SEQ ID NO: 8, (ii) covers >90% of the length of the matching protein, and (iii) has >50% identity with SEQ ID NO: 8 (when optimally aligned using the parameters provided herein). In a further embodiment, the PntB transhydrogenase is at least 95% identical to SEQ ID NO: 9. In yet another embodiment, the PntB transhydrogenase enzyme refers to an enzyme with the amino acid sequence of SEQ ID NO: 9 or a homolog thereof, wherein a PntB transhydrogenase homolog is a protein whose BLAST alignment (i) covers >90% length of SEQ ID NO: 9, (ii) covers >90% of the length of the matching protein, and (iii) has >50% identity with SEQ ID NO: 9 (when optimally aligned using the parameters provided herein). In a further embodiment, the PntAB transhydrogenase comprises a sequence at least 95% identical to SEQ ID NO: 8 and further comprises a sequence at least 95% identical to SEQ ID NO: 9. In yet another embodiment, the PntAB transhydrogenase enzyme refers to an enzyme with the amino acid sequence of both SEQ ID NO: 8 and SEQ ID NO: 9 or a homolog thereof, wherein a PntAB transhydrogenase homolog is a protein whose BLAST alignment (i) covers >90% length of SEQ ID NO: 8 and covers >90% length of SEQ ID NO: 9, (ii) covers >90% of the length of the matching protein, and (iii) has >50% identity with SEQ ID NO: 8, and has >50% identity with SEQ ID NO: 9 (when optimally aligned using the parameters provided herein).

In addition, the present invention provides an engineered photosynthetic microbe, wherein the engineered photosynthetic microbe comprises a recombinant NADPH-generating pyruvate dehydrogenase. In one embodiment, the recombinant NADPH-generating pyruvate dehydrogenase is Euglena gracilis Pno or Cryptosporidium parvum Pno. In another embodiment, the recombinant NADPH-generating pyruvate dehydrogenase is at least 95% identical to SEQ ID NO: 10. In yet another embodiment, the recombinant NADPH-generating pyruvate dehydrogenase enzyme refers to an enzyme with the amino acid sequence of SEQ ID NO: 10 or a homolog thereof, wherein a recombinant NADPH-generating pyruvate dehydrogenase homolog is a protein whose BLAST alignment (i) covers >90% length of SEQ ID NO: 10, (ii) covers >90% of the length of the matching protein, and (iii) has >50% identity with SEQ ID NO: 10 (when optimally aligned using the parameters provided herein). In still another embodiment, the recombinant NADPH-generating pyruvate dehydrogenase is at least 95% identical to SEQ ID NO: 11. In yet another embodiment, the recombinant NADPH-generating pyruvate dehydrogenase enzyme refers to an enzyme with the amino acid sequence of SEQ ID NO: 11 or a homolog thereof, wherein a recombinant NADPH-generating pyruvate dehydrogenase homolog is a protein whose BLAST alignment (i) covers >90% length of SEQ ID NO: 11, (ii) covers >90% of the length of the matching protein, and (iii) has >50% identity with SEQ ID NO: 11 (when optimally aligned using the parameters provided herein). In a further embodiment, the engineered photosynthetic microbe naturally lacks an endogenous pyruvate dehydrogenase activity or comprises a mutation which attenuates or knocks out endogenous pyruvate dehydrogenase activity.

The present invention provides, in certain aspects, an engineered photosynthetic microbe, wherein the engineered photosynthetic microbe comprises a recombinant pyruvate:ferredoxin oxidoreductase, wherein expression of the recombinant pyruvate:ferredoxin oxidoreductase is expressed by a gene, wherein the gene is controlled by a promoter which leads to increased expression of the pyruvate:ferredoxin oxidoreductase relative to that obtained with the endogenous gene under the control of its native promoter, or wherein the gene is present in a copy number which leads to increased expression of the pyruvate:ferredoxin oxidoreductase relative to that obtained with an otherwise identical photosynthetic microbe with a lower copy number. In one embodiment, the recombinant pyruvate:ferredoxin oxidoreductase is at least 95% identical to SEQ ID NO: 12. In another embodiment, the recombinant pyruvate:ferredoxin oxidoreductase enzyme refers to an enzyme with the amino acid sequence of SEQ ID NO: 12 or a homolog thereof, wherein a recombinant pyruvate:ferredoxin oxidoreductase homolog is a protein whose BLAST alignment (i) covers >90% length of SEQ ID NO: 12, (ii) covers >90% of the length of the matching protein, and (iii) has >50% identity with SEQ ID NO: 12 (when optimally aligned using the parameters provided herein).

In other embodiments, the present invention also provides an engineered photosynthetic microbe, wherein the engineered photosynthetic microbe comprises a recombinant NADPH-generating pyruvate dehydrogenase system, wherein the recombinant NADPH-generating pyruvate dehydrogenase system comprises a pyruvate decarboxylase, an NADP-dependent acetaldehyde dehydrogenase, and an acetyl-CoA synthetase. In one embodiment, the pyruvate decarboxylase is Zymomonas mobilis pyruvate decarboxylase. In another embodiment, the pyruvate decarboxylase is at least 95% identical to SEQ ID NO: 13. In still another embodiment, the pyruvate decarboxylase enzyme refers to an enzyme with the amino acid sequence of SEQ ID NO: 13 or a homolog thereof, wherein a recombinant NADPH-generating pyruvate dehydrogenase homolog is a protein whose BLAST alignment (i) covers >90% length of SEQ ID NO: 13, (ii) covers >90% of the length of the matching protein, and (iii) has >50% identity with SEQ ID NO: 13 (when optimally aligned using the parameters provided herein). In one aspect, the NADP-dependent acetaldehyde dehydrogenase is E. coli AldB. In another aspect, the NADP-dependent acetaldehyde dehydrogenase is at least 95% identical to SEQ ID NO: 14. In still another aspect, the NADP-dependent acetaldehyde dehydrogenase refers to an enzyme with the amino acid sequence of SEQ ID NO: 14 or a homolog thereof, wherein a NADP-dependent acetaldehyde dehydrogenase homolog is a protein whose BLAST alignment (i) covers >90% length of SEQ ID NO: 14, (ii) covers >90% of the length of the matching protein, and (iii) has >50% identity with SEQ ID NO: 14 (when optimally aligned using the parameters provided herein). In another related embodiment, the acetyl-CoA synthetase is E. coli Acs. In still another related embodiment, the acetyl-CoA synthetase is at least 95% identical to SEQ ID NO: 15. In yet another related embodiment, the acetyl-CoA synthetase enzyme refers to an enzyme with the amino acid sequence of SEQ ID NO: 15 or a homolog thereof, wherein the acetyl-CoA synthetase homolog is a protein whose BLAST alignment (i) covers >90% length of SEQ ID NO: 15, (ii) covers >90% of the length of the matching protein, and (iii) has >50% identity with SEQ ID NO: 15 (when optimally aligned using the parameters provided herein).

The present invention provides, in certain embodiments, an engineered photosynthetic microbe comprising at least one recombinant gene selected from the group consisting of pyruvate decarboxylase and alcohol dehydrogenase.

In addition, the present invention provides methods for improving production of a carbon-based compound of interest by a photosynthetic microbe, wherein the carbon-based compound of interest is synthesized by the photosynthetic microbe using pyruvate, at least in part, as a source of carbon, comprising: (a) culturing the photosynthetic microbe in the presence of light and an inorganic carbon source, and (b) recombinantly expressing an MdhP enzyme in the photosynthetic microbe. In one embodiment, the recombinant expression of the MdhP enzyme in the photosynthetic microbe results in increased carbon flux to pyruvate in the photosynthetic microbe. In another embodiment, the photosynthetic microbe comprises an additional mutation which reduces the expression or activity of its endogenous Mdh enzyme. In a further embodiment, the mutation is a knockout of the gene encoding the endogenous Mdh enzyme. In another aspect, the method comprises recombinantly expressing a phosphoenolpyruvate carboxylase enzyme. In still another aspect, the method comprises recombinantly expressing a recombinant NADPH-linked malic enzyme. In yet another aspect, the method comprises recombinantly expressing a phosphoenolpyruvate carboxylase enzyme and an NADPH-linked malic enzyme. In one embodiment of the method, the recombinant expression results in increased carbon flux to pyruvate in the photosynthetic microbe.

The present invention also provides methods for improving production of a carbon-based compound of interest by a photosynthetic microbe, wherein the carbon-based compound of interest is synthesized by the photosynthetic microbe using pyruvate, at least in part, as a source of carbon, comprising: (a) culturing the photosynthetic microbe in the presence of light and an inorganic carbon source, and (b) recombinantly expressing an oxaloacetate decarboxylase enzyme in the photosynthetic microbe. In one embodiment, the recombinant expression of the oxaloacetate decarboxylase enzyme in the photosynthetic microbe results in increased carbon flux to pyruvate in the photosynthetic microbe. In another embodiment of the method, the photosynthetic microbe comprises a recombinant phosphoenol pyruvate carboxylase. In one aspect, the method further comprises recombinantly expressing a phosphoenolpyruvate carboxykinase in the photosynthetic microbe. In one embodiment, the photosynthetic microbe lacks an endogenous or recombinant malate dehydrogenase activity, or wherein the engineered photosynthetic microbe comprises a mutation which attenuates or knocks out endogenous malate dehydrogenase activity in the engineered photosynthetic microbe. In another embodiment, the engineered photosynthetic microbe comprises a mutation which attenuates or knocks out endogenous pyruvate dehydrogenase activity in the photosynthetic microbe.

The present invention provides, in certain embodiments, a method for improving production of a carbon-based compound of interest by a photosynthetic microbe, wherein the carbon-based compound of interest is synthesized by the photosynthetic microbe using acetyl-CoA, at least in part, as a source of carbon, comprising: (a) culturing the photosynthetic microbe in the presence of light and an inorganic carbon source, and (b) recombinantly expressing an NADPH-producing transhydrogenase system in the photosynthetic microbe. In one embodiment, the recombinant expression of the NADPH-producing transhydrogenase in the photosynthetic microbe results in increased carbon flux to acetyl-CoA in the photosynthetic microbe.

In addition, the present invention provides methods for improving production of a carbon-based compound of interest by a photosynthetic microbe, wherein the carbon-based compound of interest is synthesized by the photosynthetic microbe using acetyl-CoA, at least in part, as a source of carbon, comprising: (a) culturing the photosynthetic microbe in the presence of light and an inorganic carbon source, and (b) recombinantly expressing an NADPH-generating pyruvate dehydrogenase in the photosynthetic microbe. In one aspect, the recombinant expression of the NADPH-generating pyruvate dehydrogenase in the photosynthetic microbe results in increased carbon flux to acetyl-CoA in the photosynthetic microbe. In another aspect, the photosynthetic microbe naturally lacks an endogenous pyruvate dehydrogenase activity or comprises a mutation which attenuates or knocks out endogenous pyruvate dehydrogenase activity.

The present invention further provides a method for improving production of a carbon-based compound of interest by a photosynthetic microbe, wherein the carbon-based compound of interest is synthesized by the photosynthetic microbe using acetyl-CoA, at least in part, as a source of carbon, comprising: (a) culturing the photosynthetic microbe in the presence of light and an inorganic carbon source, and (b) recombinantly expressing a pyruvate:ferredoxin oxidoreductase in the photosynthetic microbe, wherein expression of the recombinant pyruvate:ferredoxin oxidoreductase is expressed by a gene, wherein the gene is controlled by a promoter which leads to increased expression of the pyruvate:ferredoxin oxidoreductase relative to that obtained with the endogenous gene under the control of its native promoter, or wherein the gene is present in a copy number which leads to increased expression of the pyruvate:ferredoxin oxidoreductase relative to that obtained with an otherwise identical photosynthetic microbe with a lower copy number. In one embodiment, the recombinant expression of the pyruvate:ferredoxin oxidoreductase in the photosynthetic microbe results in increased carbon flux to acetyl-CoA in the photosynthetic microbe.

The present invention provides, in certain embodiments, a method for improving production of a carbon-based compound of interest by a photosynthetic microbe, wherein the carbon-based compound of interest is synthesized by the photosynthetic microbe using acetyl-CoA, at least in part, as a source of carbon, comprising: (a) culturing the photosynthetic microbe in the presence of light and an inorganic carbon source, and (b) recombinantly expressing an NADPH-generating pyruvate dehydrogenase system in the photosynthetic microbe, wherein the NADPH-generating pyruvate dehydrogenase system comprises a pyruvate decarboxylase, an NADP-dependent acetaldehyde dehydrogenase, and an acetyl-CoA synthetase. In one aspect, the recombinant expression of the NADPH-generating pyruvate dehydrogenase system in the photosynthetic microbe results in increased carbon flux to acetyl-CoA in the photosynthetic microbe.

In some embodiments, the carbon-based compound of interest is produced at a greater rate or in greater yields in the engineered photosynthetic microbe relative to an otherwise identical photosynthetic microbe lacking the recited recombinant enzymes or mutations.

In one embodiment, the engineered photosynthetic microbe comprises at least one recombinant gene selected from the group consisting of pyruvate decarboxylase and alcohol dehydrogenase. In related embodiments, the carbon-based compound of interest is ethanol. In another related embodiment, the carbon-based compound of interest is selected from the group consisting of: alcohols, alkenes, and alkanes.

In one embodiment, a heterotrophic organism is used for the above embodiments of the invention instead of a photosynthetic microbe.

These and other embodiments of the invention are further described in the Figures, Description, Examples and Claims, herein.

### BRIEF DESCRIPTION OF THE FIGURES

FIG. 1 depicts an enzymatic pathway for the biosynthesis of pyruvate and/or acetyl-CoA from endogenous enzymatic activity. Both autotrophic and heterotrophic pyruvate formation pathways are shown. 1,3-BPG=1,3-bisphosphoglycerate. 3-PG=3-phosphoglycerate. 2-PG=2-phosphoglycerate. PEP=phosphoenolpyruvate. OAA=oxaloacetic acid. pgk encodes phosphoglycerate kinase. pgm encodes phosphoglycerate mutase. eno encodes enolase (phosphopyruvate hydratase). ppc encodes phosphoenolpyruvate carboxylase. mdh encodes malate dehydrogenase. maeB encodes NADP-dependent malic enzyme. pyk encodes pyruvate kinase. pdh (e.g., aceEF-lpd) encodes pyruvate dehydrogenase.

FIG. 2 depicts an alternative recombinant malate biosynthesis pathway utilizing an NADPH-dependent malate dehydrogenase (encoded by the mdhP gene) (underlined) to synthesize malate from oxaloacetate. pdh (pyruvate dehydrogenase) is optionally attenuated to prevent usage of pyruvate to form acetyl-CoA.

FIG. 3 depicts an alternative recombinant pyruvate biosynthesis pathway from phosphoenolpyruvate. The biosynthesis pathway includes NADPH-dependent malate dehydrogenase (encoded by the mdhP gene) enzyme (underlined) to convert oxaloacetic acid to malate. The biosynthesis pathway optionally also includes recombinant phosphoenolpyruvate carboxylase (encoded by the ppc gene) and NADP-dependent malic enzyme (encoded by the maeB gene) (underlined) to biosynthesize oxaloacetic acid from phosphoenolpyruvate, and pyruvate from malate, respectively. pdh (pyruvate dehydrogenase) is optionally attenuated to prevent usage of pyruvate to form acetyl-CoA.

FIG. 4 depicts an alternative recombinant pathway for biosynthesis of pyruvate from oxaloacetic acid using recombinant oxaloacetate decarboxylase (encoded by the odx gene) (underlined). Phosphoenolpyruvate carboxykinase (encoded by the pck gene) (underlined) may also be used to enhance the rate of biosynthesis of oxaloacetic acid (OAA) from phosphoenol pyruvate (PEP). pdh is optionally attenuated to prevent usage of pyruvate to form acetyl-CoA

FIG. 5 depicts several optional recombinant pathways (underlined) for increased biosynthesis of pyruvate without using NADH. pdh (pyruvate dehydrogenase) is optionally attenuated to prevent usage of pyruvate to form acetyl-CoA.

FIG. 6 depicts a pathway for biosynthesis of acetyl-CoA from pyruvate comprising recombinant proton-translocating transhydrogenase (encoded by the pntAB gene) (underlined) to convert excess NADH to NADPH.

FIG. 7 depicts an alternative recombinant pathway for biosynthesis of acetyl-CoA from pyruvate using recombinant NADP+-dependent oxidoreductase (encoded by the pno gene) (underlined) to convert pyruvate to acetyl-CoA. pdh (pyruvate dehydrogenase) is optionally attenuated to decrease NADH production.

FIG. 8 depicts an alternative recombinant pathway for biosynthesis of acetyl-CoA from pyruvate using recombinant pyruvate:ferredoxin oxidoreductase (PFO, encoded by the nig gene) (underlined) to convert pyruvate to acetyl-CoA. pdh (pyruvate dehydrogenase) is optionally attenuated to decrease NADH production.

FIG. 9 depicts an alternative recombinant pathway for biosynthesis of acetyl-CoA from pyruvate via acetaldehyde and acetate based on the sequential activity of recombinant pyruvate decarboxylase (encoded by the pdc gene), aldehyde dehydrogenase (encoded by the aldB gene), and acetaldehyde dehydrogenase (encoded by the acs gene) (underlined) to convert pyruvate to acetaldehyde, acetaldehyde to acetate, and acetate to acetyl-CoA, respectively. pdh (pyruvate dehydrogenase) is optionally attenuated to decrease NADH production.

### DETAILED DESCRIPTION OF THE INVENTION

Unless otherwise defined herein, scientific and technical terms used in connection with the present invention shall have the meanings that are commonly understood by those of ordinary skill in the art. Further, unless otherwise required by context, singular terms shall include the plural and plural terms shall include the singular. Generally, nomenclatures used in connection with, and techniques of, biochemistry, enzymology, molecular and cellular biology, microbiology, genetics and protein and nucleic acid chemistry and hybridization described herein are those well known and commonly used in the art.

The methods and techniques of the present invention are generally performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol I, CRC Press (1976); Handbook of Biochemistry: Section A Proteins, Vol II, CRC Press (1976); Essentials of Glycobiology, Cold Spring Harbor Laboratory Press (1999).

All publications, patents and other references mentioned herein are hereby incorporated by reference in their entireties.

The following terms, unless otherwise indicated, shall be understood to have the following meanings:

The term “polynucleotide” or “nucleic acid molecule” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation.

Unless otherwise indicated, and as an example for all sequences described herein under the general format “SEQ ID NO:”, “nucleic acid comprising SEQ ID NO:1” refers to a nucleic acid, at least a portion of which has either (i) the sequence of SEQ ID NO:1, or (ii) a sequence complementary to SEQ ID NO:1. The choice between the two is dictated by the context. For instance, if the nucleic acid is used as a probe, the choice between the two is dictated by the requirement that the probe be complementary to the desired target.

An “isolated” RNA, DNA or a mixed polymer is one which is substantially separated from other cellular components that naturally accompany the native polynucleotide in its natural host cell, e.g., ribosomes, polymerases and genomic sequences with which it is naturally associated.

As used herein, an “isolated” organic molecule (e.g., an alkane, alkene, or alkanal) is one which is substantially separated from the cellular components (membrane lipids, chromosomes, proteins) of the host cell from which it originated, or from the medium in which the host cell was cultured. The term does not require that the biomolecule has been separated from all other chemicals, although certain isolated biomolecules may be purified to near homogeneity.

The term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

As used herein, an endogenous nucleic acid sequence in the genome of an organism (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “recombinant” because it is separated from at least some of the sequences that naturally flank it.

A nucleic acid is also considered “recombinant” if it contains any modifications that do not naturally occur to the corresponding nucleic acid in a genome. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome.

As used herein, the phrase “degenerate variant” of a reference nucleic acid sequence encompasses nucleic acid sequences that can be translated, according to the standard genetic code, to provide an amino acid sequence identical to that translated from the reference nucleic acid sequence. The term “degenerate oligonucleotide” or “degenerate primer” is used to signify an oligonucleotide capable of hybridizing with target nucleic acid sequences that are not necessarily identical in sequence but that are homologous to one another within one or more particular segments.

The term “percent sequence identity” or “identical” in the context of nucleic acid sequences refers to the residues in the two sequences which are the same when aligned for maximum correspondence. The length of sequence identity comparison may be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA provides alignments and percent sequence identity of the regions of the best overlap between the query and search sequences. Pearson, Methods Enzymol. 183:63-98 (1990) (hereby incorporated by reference in its entirety). For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference. Alternatively, sequences can be compared using the computer program, BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

The term “substantial homology” or “substantial similarity,” when referring to a nucleic acid or fragment thereof, indicates that, when optimally aligned with appropriate nucleotide insertions or deletions with another nucleic acid (or its complementary strand), there is nucleotide sequence identity in at least about 76%, 80%, 85%, preferably at least about 90%, and more preferably at least about 95%, 96%, 97%, 98% or 99% of the nucleotide bases, as measured by any well-known algorithm of sequence identity, such as FASTA, BLAST or Gap, as discussed above.

Alternatively, substantial homology or similarity exists when a nucleic acid or fragment thereof hybridizes to another nucleic acid, to a strand of another nucleic acid, or to the complementary strand thereof, under stringent hybridization conditions. “Stringent hybridization conditions” and “stringent wash conditions” in the context of nucleic acid hybridization experiments depend upon a number of different physical parameters. Nucleic acid hybridization will be affected by such conditions as salt concentration, temperature, solvents, the base composition of the hybridizing species, length of the complementary regions, and the number of nucleotide base mismatches between the hybridizing nucleic acids, as will be readily appreciated by those skilled in the art. One having ordinary skill in the art knows how to vary these parameters to achieve a particular stringency of hybridization.

In general, “stringent hybridization” is performed at about 25° C. below the thermal melting point (Tₘ) for the specific DNA hybrid under a particular set of conditions. “Stringent washing” is performed at temperatures about 5° C. lower than the Tₘ for the specific DNA hybrid under a particular set of conditions. The Tₘ is the temperature at which 50% of the target sequence hybridizes to a perfectly matched probe. See Sambrook et al., Molecular Cloning: A Laboratory Manual, 2d ed., Cold Spring Harbor Laboratory Press, Cold Spring Harbor, N.Y. (1989), page 9.51, hereby incorporated by reference. For purposes herein, “stringent conditions” are defined for solution phase hybridization as aqueous hybridization (i.e., free of formamide) in 6×SSC (where 20×SSC contains 3.0 M NaCl and 0.3 M sodium citrate), 1% SDS at 65° C. for 8-12 hours, followed by two washes in 0.2×SSC, 0.1% SDS at 65° C. for 20 minutes. It will be appreciated by the skilled worker that hybridization at 65° C. will occur at different rates depending on a number of factors including the length and percent identity of the sequences which are hybridizing.

The nucleic acids (also referred to as polynucleotides) of this present invention may include both sense and antisense strands of RNA, cDNA, genomic DNA, and synthetic forms and mixed polymers of the above. They may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.) Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule. Other modifications can include, for example, analogs in which the ribose ring contains a bridging moiety or other structure such as the modifications found in “locked” nucleic acids.

The term “mutated” when applied to nucleic acid sequences means that nucleotides in a nucleic acid sequence may be inserted, deleted or changed compared to a reference nucleic acid sequence. A single alteration may be made at a locus (a point mutation) or multiple nucleotides may be inserted, deleted or changed at a single locus. In addition, one or more alterations may be made at any number of loci within a nucleic acid sequence. A nucleic acid sequence may be mutated by any method known in the art including but not limited to mutagenesis techniques such as “error-prone PCR” (a process for performing PCR under conditions where the copying fidelity of the DNA polymerase is low, such that a high rate of point mutations is obtained along the entire length of the PCR product; see, e.g., Leung et al., Technique, 1:11-15 (1989) and Caldwell and Joyce, PCR Methods Applic. 2:28-33 (1992)); and “oligonucleotide-directed mutagenesis” (a process which enables the generation of site-specific mutations in any cloned DNA segment of interest; see, e.g., Reidhaar-Olson and Sauer, Science 241:53-57 (1988)).

The term “attenuate” as used herein generally refers to a functional deletion, including a mutation, partial or complete deletion, insertion, or other variation made to a gene sequence or a sequence controlling the transcription of a gene sequence, which reduces or inhibits production of the gene product, or renders the gene product non-functional. In some instances a functional deletion is described as a knockout mutation. Attenuation also includes amino acid sequence changes by altering the nucleic acid sequence, placing the gene under the control of a less active promoter, down-regulation, expressing interfering RNA, ribozymes or antisense sequences that target the gene of interest, or through any other technique known in the art. In one example, the sensitivity of a particular enzyme to feedback inhibition or inhibition caused by a composition that is not a product or a reactant (non-pathway specific feedback) is lessened such that the enzyme activity is not impacted by the presence of a compound. In other instances, an enzyme that has been altered to be less active can be referred to as attenuated.

The term “deletion” as used herein is intended to refer to the removal of one or more nucleotides from a nucleic acid molecule or one or more amino acids from a protein, the regions on either side being joined together.

The term “knock-out” as used herein is intended to refer to a gene whose level of expression or activity has been reduced to zero. In some examples, a gene is knocked-out via deletion of some or all of its coding sequence. In other examples, a gene is knocked-out via introduction of one or more nucleotides into its open reading frame, which results in translation of a non-sense or otherwise non-functional protein product.

The term “vector” as used herein is intended to refer to a nucleic acid molecule capable of transporting another nucleic acid to which it has been linked. One type of vector is a “plasmid,” which generally refers to a circular double stranded DNA loop into which additional DNA segments may be ligated, but also includes linear double-stranded molecules such as those resulting from amplification by the polymerase chain reaction (PCR) or from treatment of a circular plasmid with a restriction enzyme. Other vectors include cosmids, bacterial artificial chromosomes (BAC) and yeast artificial chromosomes (YAC). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain preferred vectors are capable of directing the expression of genes to which they are operatively linked. Such vectors are referred to herein as “recombinant expression vectors” (or simply “expression vectors”).

“Operatively linked” or “operably linked” expression control sequences refers to a linkage in which the expression control sequence is contiguous with the gene of interest to control the gene of interest, as well as expression control sequences that act in trans or at a distance to control the gene of interest.

The term “expression control sequence” as used herein refers to polynucleotide sequences which are necessary to affect the expression of coding sequences to which they are operatively linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “recombinant host cell” (or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism.

The term “peptide” as used herein refers to a short polypeptide, e.g., one that is typically less than about 50 amino acids long and more typically less than about 30 amino acids long. The term as used herein encompasses analogs and mimetics that mimic structural and thus biological function.

The term “polypeptide” encompasses both naturally-occurring and non-naturally-occurring proteins, and fragments, mutants, derivatives and analogs thereof. A polypeptide may be monomeric or polymeric. Further, a polypeptide may comprise a number of different domains each of which has one or more distinct activities.

The term “isolated protein” or “isolated polypeptide” is a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art. As thus defined, “isolated” does not necessarily require that the protein, polypeptide, peptide or oligopeptide so described has been physically removed from its native environment.

The term “polypeptide fragment” as used herein refers to a polypeptide that has a deletion, e.g., an amino-terminal and/or carboxy-terminal deletion compared to a full-length polypeptide. In a preferred embodiment, the polypeptide fragment is a contiguous sequence in which the amino acid sequence of the fragment is identical to the corresponding positions in the naturally-occurring sequence. Fragments typically are at least 5, 6, 7, 8, 9 or 10 amino acids long, preferably at least 12, 14, 16 or 18 amino acids long, more preferably at least 20 amino acids long, more preferably at least 25, 30, 35, 40 or 45, amino acids, even more preferably at least 50 or 60 amino acids long, and even more preferably at least 70 amino acids long.

A “modified derivative” refers to polypeptides or fragments thereof that are substantially homologous in primary structural sequence but which include, e.g., in vivo or in vitro chemical and biochemical modifications or which incorporate amino acids that are not found in the native polypeptide. Such modifications include, for example, acetylation, carboxylation, phosphorylation, glycosylation, ubiquitination, labeling, e.g., with radionuclides, and various enzymatic modifications, as will be readily appreciated by those skilled in the art. A variety of methods for labeling polypeptides and of substituents or labels useful for such purposes are well known in the art, and include radioactive isotopes such as ¹²⁵I, ³²P, ³⁵_{S, and} ³H, ligands which bind to labeled antiligands (e.g., antibodies), fluorophores, chemiluminescent agents, enzymes, and antiligands which can serve as specific binding pair members for a labeled ligand. The choice of label depends on the sensitivity required, ease of conjugation with the primer, stability requirements, and available instrumentation. Methods for labeling polypeptides are well known in the art. See, e.g., Ausubel et al., Current Protocols in Molecular Biology, Greene Publishing Associates (1992, and Supplements to 2002) (hereby incorporated by reference).

The term “fusion protein” refers to a polypeptide comprising a polypeptide or fragment coupled to heterologous amino acid sequences. Fusion proteins are useful because they can be constructed to contain two or more desired functional elements from two or more different proteins. A fusion protein comprises at least 10 contiguous amino acids from a polypeptide of interest, more preferably at least 20 or 30 amino acids, even more preferably at least 40, 50 or 60 amino acids, yet more preferably at least 75, 100 or 125 amino acids. Fusions that include the entirety of the proteins of the present invention have particular utility. The heterologous polypeptide included within the fusion protein of the present invention is at least 6 amino acids in length, often at least 8 amino acids in length, and usefully at least 15, 20, and 25 amino acids in length. Fusions that include larger polypeptides, such as an IgG Fc region, and even entire proteins, such as the green fluorescent protein (“GFP”) chromophore-containing proteins, have particular utility. Fusion proteins can be produced recombinantly by constructing a nucleic acid sequence which encodes the polypeptide or a fragment thereof in frame with a nucleic acid sequence encoding a different protein or peptide and then expressing the fusion protein. Alternatively, a fusion protein can be produced chemically by crosslinking the polypeptide or a fragment thereof to another protein.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives.

Fragments within the scope of the term “antibody” include those produced by digestion with various proteases, those produced by chemical cleavage and/or chemical dissociation and those produced recombinantly, so long as the fragment remains capable of specific binding to a target molecule. Among such fragments are Fab, Fab′, Fv, F(ab′).sub.2, and single chain Fv (scFv) fragments.

Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Intracellular Antibodies: Research and Disease Applications, (Marasco, ed., Springer-Verlag New York, Inc., 1998), the disclosure of which is incorporated herein by reference in its entirety).

As used herein, antibodies can be produced by any known technique, including harvest from cell culture of native B lymphocytes, harvest from culture of hybridomas, recombinant expression systems and phage display.

The term “non-peptide analog” refers to a compound with properties that are analogous to those of a reference polypeptide. A non-peptide compound may also be termed a “peptide mimetic” or a “peptidomimetic.” See, e.g., Jones, Amino Acid and Peptide Synthesis, Oxford University Press (1992); Jung, Combinatorial Peptide and Nonpeptide Libraries: A Handbook, John Wiley (1997); Bodanszky et al., Peptide Chemistry—A Practical Textbook, Springer Verlag (1993); Synthetic Peptides: A Users Guide, (Grant, ed., W. H. Freeman and Co., 1992); Evans et al., J. Med. Chem. 30:1229 (1987); Fauchere, J. Adv. Drug Res. 15:29 (1986); Veber and Freidinger, Trends Neurosci., 8:392-396 (1985); and references sited in each of the above, which are incorporated herein by reference. Such compounds are often developed with the aid of computerized molecular modeling. Peptide mimetics that are structurally similar to useful peptides of the present invention may be used to produce an equivalent effect and are therefore envisioned to be part of the present invention.

A “polypeptide mutant” or “mutein” refers to a polypeptide whose sequence contains an insertion, duplication, deletion, rearrangement or substitution of one or more amino acids compared to the amino acid sequence of a native or wild-type protein. A mutein may have one or more amino acid point substitutions, in which a single amino acid at a position has been changed to another amino acid, one or more insertions and/or deletions, in which one or more amino acids are inserted or deleted, respectively, in the sequence of the naturally-occurring protein, and/or truncations of the amino acid sequence at either or both the amino or carboxy termini. A mutein may have the same but preferably has a different biological activity compared to the naturally-occurring protein.

A mutein has at least 85% overall sequence homology to its wild-type counterpart. Even more preferred are muteins having at least 90% overall sequence homology to the wild-type protein.

In an even more preferred embodiment, a mutein exhibits at least 95% sequence identity, even more preferably 98%, even more preferably 99% and even more preferably 99.9% overall sequence identity.

Sequence homology may be measured by any common sequence analysis algorithm, such as Gap or Bestfit.

Amino acid substitutions can include those which: (1) reduce susceptibility to proteolysis, (2) reduce susceptibility to oxidation, (3) alter binding affinity for forming protein complexes, (4) alter binding affinity or enzymatic activity, and (5) confer or modify other physicochemical or functional properties of such analogs.

As used herein, the twenty conventional amino acids and their abbreviations follow conventional usage. See Immunology—A Synthesis (Golub and Gren eds., Sinauer Associates, Sunderland, Mass., 2^{nd} ed. 1991), which is incorporated herein by reference. Stereoisomers (e.g., D-amino acids) of the twenty conventional amino acids, unnatural amino acids such as α-, α-disubstituted amino acids, N-alkyl amino acids, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, N-methylarginine, and other similar amino acids and imino acids (e.g., 4-hydroxyproline). In the polypeptide notation used herein, the left-hand end corresponds to the amino terminal end and the right-hand end corresponds to the carboxy-terminal end, in accordance with standard usage and convention.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences.) As used herein, homology between two regions of amino acid sequence (especially with respect to predicted structural similarities) is interpreted as implying similarity in function.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art. See, e.g., Pearson, 1994, Methods Mol. Biol. 24:307-31 and 25:365-89 (herein incorporated by reference).

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using a measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild-type protein and a mutein thereof. See, e.g., GCG Version 6.1.

A preferred algorithm when comparing a particular polypeptide sequence to a database containing a large number of sequences from different organisms is the computer program BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences. Pearson, Methods Enzymol. 183:63-98 (1990) (incorporated by reference herein). For example, percent sequence identity between amino acid sequences can be determined using FASTA with its default parameters (a word size of 2 and the PAM250 scoring matrix), as provided in GCG Version 6.1, herein incorporated by reference.

“Specific binding” refers to the ability of two molecules to bind to each other in preference to binding to other molecules in the environment. Typically, “specific binding” discriminates over adventitious binding in a reaction by at least two-fold, more typically by at least 10-fold, often at least 100-fold. Typically, the affinity or avidity of a specific binding reaction, as quantified by a dissociation constant, is about 10⁻⁷ M or stronger (e.g., about 10⁻⁸ M, 10⁻⁹ M or even stronger).

“Percent dry cell weight” refers to a measurement of carbon-based compound of interest, (e.g., hydrocarbon) production obtained as follows: a defined volume of culture is centrifuged to pellet the cells. Cells are washed then dewetted by at least one cycle of microcentrifugation and aspiration. Cell pellets are lyophilized overnight, and the tube containing the dry cell mass is weighed again such that the mass of the cell pellet can be calculated within ±0.1 mg. At the same time cells are processed for dry cell weight determination, a second sample of the culture in question is harvested, washed, and dewetted. The resulting cell pellet, corresponding to 1-3 mg of dry cell weight, is then extracted by vortexing in approximately 1 ml acetone plus butylated hydroxytolune (BHT) as antioxidant and an internal standard, e.g., n-heptacosane. Cell debris is then pelleted by centrifugation and the supernatant (extractant) is taken for analysis by GC. For accurate quantitation of a compound of interest, such as n-alkanes, flame ionization detection (FID) is used as opposed to MS total ion count. n-Alkane concentrations in the biological extracts are calculated using calibration relationships between GC-FID peak area and known concentrations of authentic n-alkane standards. Knowing the volume of the extractant, the resulting concentrations of the n-alkane species in the extracant, and the dry cell weight of the cell pellet extracted, the percentage of dry cell weight that comprised n-alkanes can be determined.

The term “region” as used herein refers to a physically contiguous portion of the primary structure of a biomolecule. In the case of proteins, a region is defined by a contiguous portion of the amino acid sequence of that protein.

The term “domain” as used herein refers to a structure of a biomolecule that contributes to a known or suspected function of the biomolecule. Domains may be co-extensive with regions or portions thereof; domains may also include distinct, non-contiguous regions of a biomolecule. Examples of protein domains include, but are not limited to, an Ig domain, an extracellular domain, a transmembrane domain, and a cytoplasmic domain.

As used herein, the term “molecule” means any compound, including, but not limited to, a small molecule, peptide, protein, sugar, nucleotide, nucleic acid, lipid, etc., and such a compound can be natural or synthetic.

“Carbon-based Compounds of Interest” include alcohols such as ethanol, propanol, isopropanol, butanol, fatty alcohols, fatty acid esters, wax esters; hydrocarbons and alkanes such as propane, octane, diesel, Jet Propellant 8 (JP8); polymers such as terephthalate, 1,3-propanediol, 1,4-butanediol, polyols, Polyhydroxyalkanoates (PHA), poly-beta-hydroxybutyrate (PHB), acrylate, adipic acid, E-caprolactone, isoprene, caprolactam, rubber; commodity chemicals such as lactate, Docosahexaenoic acid (DHA), 3-hydroxypropionate, γ-valerolactone, lysine, serine, aspartate, aspartic acid, sorbitol, ascorbate, ascorbic acid, isopentenol, lanosterol, omega-3 DHA, lycopene, itaconate, 1,3-butadiene, ethylene, propylene, succinate, citrate, citric acid, glutamate, malate, 3-hydroxypropionic acid (HPA), lactic acid, THF, gamma butyrolactone, pyrrolidones, hydroxybutyrate, glutamic acid, levulinic acid, acrylic acid, malonic acid; specialty chemicals such as carotenoids, isoprenoids, itaconic acid; pharmaceuticals and pharmaceutical intermediates such as 7-aminodeacetoxycephalosporanic acid (7-ADCA)/cephalosporin, erythromycin, polyketides, statins, paclitaxel, docetaxel, terpenes, peptides, steroids, omega fatty acids and other such suitable products of interest. Such products are useful in the context of biofuels, industrial and specialty chemicals, as intermediates used to make additional products, such as nutritional supplements, neutraceuticals, polymers, paraffin replacements, personal care products and pharmaceuticals.

Biofuel: A biofuel refers to any fuel that derives from a biological source. Biofuel can refer to one or more hydrocarbons, one or more alcohols, one or more fatty esters or a mixture thereof.

Hydrocarbon: The term generally refers to a chemical compound that consists of the elements carbon (C), hydrogen (H) and optionally oxygen (O). There are essentially three types of hydrocarbons, e.g., aromatic hydrocarbons, saturated hydrocarbons and unsaturated hydrocarbons such as alkenes, alkynes, and dienes. The term also includes fuels, biofuels, plastics, waxes, solvents and oils. Hydrocarbons encompass biofuels, as well as plastics, waxes, solvents and oils.

Unless otherwise defined, all technical and scientific terms used herein have the same meaning as commonly understood by one of ordinary skill in the art to which this present invention pertains. Exemplary methods and materials are described below, although methods and materials similar or equivalent to those described herein can also be used in the practice of the present invention and will be apparent to those of skill in the art. All publications and other references mentioned herein are incorporated by reference in their entirety. In case of conflict, the present specification, including definitions, will control. The materials, methods, and examples are illustrative only and not intended to be limiting.

Throughout this specification and claims, the word “comprise” or variations such as “comprises” or “comprising”, will be understood to imply the inclusion of a stated integer or group of integers but not the exclusion of any other integer or group of integers.

### Enzymatic Activity

As is well known in the art, enzyme activities can be measured in various ways. For example, the pyrophosphorolysis of OMP may be followed spectroscopically (Grubmeyer et al., (1993) J. Biol. Chem. 268:20299-20304). Alternatively, the activity of the enzyme can be followed using chromatographic techniques, such as by high performance liquid chromatography (Chung and Sloan, (1986) J. Chromatogr. 371:71-81). As another alternative the activity can be indirectly measured by determining the levels of product made from the enzyme activity. These levels can be measured with techniques including aqueous chloroform/methanol extraction as known and described in the art (Cf M. Kates (1986) Techniques of Lipidology; Isolation, analysis and identification of Lipids. Elsevier Science Publishers, New York (ISBN: 0444807322)). More modern techniques include using gas chromatography linked to mass spectrometry (Niessen, W. M. A. (2001). Current practice of gas chromatography—mass spectrometry. New York, N.Y.: Marcel Dekker. (ISBN: 0824704738)). Additional modern techniques for identification of recombinant protein activity and products including liquid chromatography-mass spectrometry (LCMS), high performance liquid chromatography (HPLC), capillary electrophoresis, Matrix-Assisted Laser Desorption Ionization time of flight-mass spectrometry (MALDI-TOF MS), nuclear magnetic resonance (NMR), near-infrared (NIR) spectroscopy, viscometry (Knothe, G (1997) Am. Chem. Soc. Symp. Series, 666: 172-208), titration for determining free fatty acids (Komers (1997) Fett/Lipid, 99(2): 52-54), enzymatic methods (Bailer (1991) Fresenius J. Anal. Chem. 340(3): 186), physical property-based methods, wet chemical methods, etc. can be used to analyze the levels and the identity of the product produced by the organisms of the present invention. Other methods and techniques may also be suitable for the measurement of enzyme activity, as would be known by one of skill in the art.

### Host Cell Transformants

In another aspect of the present invention, host cells transformed with the nucleic acid molecules or vectors of the present invention, and descendants thereof, are provided. In some embodiments of the present invention, these cells carry the nucleic acid sequences of the present invention on vectors, which may but need not be freely replicating vectors. In other embodiments of the present invention, the nucleic acids have been integrated into the genome of the host cells.

### Selected or Engineered Microorganisms for the Production of Carbon-Based Compounds of Interest

Microorganism: Includes prokaryotic and eukaryotic microbial species from the Domains Archaea, Bacteria and Eucarya, the latter including yeast and filamentous fungi, protozoa, algae, or higher Protista. l The terms “microbial cells” and “microbes” are used interchangeably with the term microorganism.

A variety of host organisms can be transformed to produce a product of interest. Photoautotrophic organisms include eukaryotic plants and algae, as well as prokaryotic cyanobacteria, green-sulfur bacteria, green non-sulfur bacteria, purple sulfur bacteria, and purple non-sulfur bacteria.

Extremophiles are also contemplated as suitable organisms. Such organisms withstand various environmental parameters such as temperature, radiation, pressure, gravity, vacuum, desiccation, salinity, pH, oxygen tension, and chemicals. They include hyperthermophiles, which grow at or above 80° C. such as Pyrolobus fumarii; thermophiles, which grow between 60-80° C. such as Synechococcus lividis; mesophiles, which grow between 15-60° C. and psychrophiles, which grow at or below 15° C. such as Psychrobacter and some insects. Radiation tolerant organisms include Deinococcus radiodurans. Pressure-tolerant organisms include piezophiles, which tolerate pressure of 130 MPa. Weight-tolerant organisms include barophiles. Hypergravity (e.g., >1 g) hypogravity (e.g., <1 g) tolerant organisms are also contemplated. Vacuum tolerant organisms include tardigrades, insects, microbes and seeds. Dessicant tolerant and anhydrobiotic organisms include xerophiles such as Artemia salina; nematodes, microbes, fungi and lichens. Salt-tolerant organisms include halophiles (e.g., 2-5 M NaCl) Halobacteriacea and Dunaliella salina. pH-tolerant organisms include alkaliphiles such as Natronobacterium, Bacillus firmus OF4, Spirulina spp. (e.g., pH >9) and acidophiles such as Cyanidium caldarium, Ferroplasma sp. (e.g., low pH). Anaerobes, which cannot tolerate O₂ such as Methanococcus jannaschii; microaerophils, which tolerate some O₂ such as Clostridium and aerobes, which require O₂ are also contemplated. Gas-tolerant organisms, which tolerate pure CO₂ include Cyanidium caldarium and metal tolerant organisms include metalotolerants such as Ferroplasma acidarmanus (e.g., Cu, As, Cd, Zn), Ralstonia sp. CH34 (e.g., Zn, Co, Cd, Hg, Pb). Gross, Michael. Life on the Edge: Amazing Creatures Thriving in Extreme Environments. New YorK: Plenum (1998) and Seckbach, J. “Search for Life in the Universe with Terrestrial Microbes Which Thrive Under Extreme Conditions.” In Cristiano Batalli Cosmovici, Stuart Bowyer, and Dan Wertheimer, eds., Astronomical and Biochemical Origins and the Search for Life in the Universe, p. 511. Milan: Editrice Compositori (1997).

Plants include but are not limited to the following genera: Arabidopsis, Beta, Glycine, Jatropha, Miscanthus, Panicum, Phalaris, Populus, Saccharum, Salix, Simmondsia and Zea.

Algae and cyanobacteria include but are not limited to the following genera: Acanthoceras, Acanthococcus, Acaryochloris, Achnanthes, Achnanthidium, Actinastrum, Actinochloris, Actinocyclus, Actinotaenium, Amphichrysis, Amphidinium, Amphikrikos, Amphipleura, Amphiprora, Amphithrix, Amphora, Anabaena, Anabaenopsis, Aneumastus, Ankistrodesmus, Ankyra, Anomoeoneis, Apatococcus, Aphanizomenon, Aphanocapsa, Aphanochaete, Aphanothece, Apiocystis, Apistonema, Arthrodesmus, Artherospira, Ascochloris, Asterionella, Asterococcus, Audouinella, Aulacoseira, Bacillaria, Balbiania, Bambusina, Bangia, Basichlamys, Batrachospermum, Binuclearia, Bitrichia, Blidingia, Botrdiopsis, Botrydium, Botryococcus, Botryosphaerella, Brachiomonas, Brachysira, Brachytrichia, Brebissonia, Bulbochaete, Bumilleria, Bumilleriopsis, Caloneis, Calothrix, Campylodiscus, Capsosiphon, Carteria, Catena, Cavinula, Centritractus, Centronella, Ceratium, Chaetoceros, Chaetochloris, Chaetomorpha, Chaetonella, Chaetonema, Chaetopeltis, Chaetophora, Chaetosphaeridium, Chamaesiphon, Chara, Characiochloris, Characiopsis, Characium, Charales, Chilomonas, Chlainomonas, Chlamydoblepharis, Chlamydocapsa, Chlamydomonas, Chlamydomonopsis, Chlamydomyxa, Chlamydonephris, Chlorangiella, Chlorangiopsis, Chlorella, Chlorobotrys, Chlorobrachis, Chlorochytrium, Chlorococcum, Chlorogloea, Chlorogloeopsis, Chlorogonium, Chlorolobion, Chloromonas, Chlorophysema, Chlorophyta, Chlorosaccus, Chlorosarcina, Choricystis, Chromophyton, Chromulina, Chroococcidiopsis, Chroococcus, Chroodactylon, Chroomonas, Chroothece, Chrysamoeba, Chrysapsis, Chrysidiastrum, Chrysocapsa, Chrysocapsella, Chrysochaete, Chrysochromulina, Chrysococcus, Chrysocrinus, Chrysolepidomonas, Chrysolykos, Chrysonebula, Chrysophyta, Chrysopyxis, Chrysosaccus, Chrysophaerella, Chrysostephanosphaera, Clodophora, Clastidium, Closteriopsis, Closterium, Coccomyxa, Cocconeis, Coelastrella, Coelastrum, Coelosphaerium, Coenochloris, Coenococcus, Coenocystis, Colacium, Coleochaete, Collodictyon, Compsogonopsis, Compsopogon, Conjugatophyta, Conochaete, Coronastrum, Cosmarium, Cosmioneis, Cosmocladium, Crateriportula, Craticula, Crinalium, Crucigenia, Crucigeniella, Cryptoaulax, Cryptomonas, Cryptophyta, Ctenophora, Cyanodictyon, Cyanonephron, Cyanophora, Cyanophyta, Cyanothece, Cyanothomonas, Cyclonexis, Cyclostephanos, Cyclotella, Cylindrocapsa, Cylindrocystis, Cylindrospermum, Cylindrotheca, Cymatopleura, Cymbella, Cymbellonitzschia, Cystodinium Dactylococcopsis, Debarya, Denticula, Dermatochrysis, Dermocarpa, Dermocarpella, Desmatractum, Desmidium, Desmococcus, Desmonema, Desmosiphon, Diacanthos, Diacronema, Diadesmis, Diatoma, Diatomella, Dicellula, Dichothrix, Dichotomococcus, Dicranochaete, Dictyochloris, Dictyococcus, Dictyosphaerium, Didymocystis, Didymogenes, Didymosphenia, Dilabifilum, Dimorphococcus, Dinobryon, Dinococcus, Diplochloris, Diploneis, Diplostauron, Distrionella, Docidium, Draparnaldia, Dunaliella, Dysmorphococcus, Ecballocystis, Elakatothrix, Ellerbeckia, Encyonema, Enteromorpha, Entocladia, Entomoneis, Entophysalis, Epichrysis, Epipyxis, Epithemia, Eremosphaera, Euastropsis, Euastrum, Eucapsis, Eucocconeis, Eudorina, Euglena, Euglenophyta, Eunotia, Eustigmatophyta, Eutreptia, Fallacia, Fischerella, Fragilaria, Fragilariforma, Franceia, Frustulia, Curcilla, Geminella, Genicularia, Glaucocystis, Glaucophyta, Glenodiniopsis, Glenodinium, Gloeocapsa, Gloeochaete, Gloeochrysis, Gloeococcus, Gloeocystis, Gloeodendron, Gloeomonas, Gloeoplax, Gloeothece, Gloeotila, Gloeotrichia, Gloiodictyon, Golenkinia, Golenkiniopsis, Gomontia, Gomphocymbella, Gomphonema, Gomphosphaeria, Gonatozygon, Gongrosia, Gongrosira, Goniochloris, Gonium, Gonyostomum, Granulochloris, Granulocystopsis, Groenbladia, Gymnodinium, Gymnozyga, Gyrosigma, Haematococcus, Hafniomonas, Hallassia, Hammatoidea, Hannaea, Hantzschia, Hapalosiphon, Haplotaenium, Haptophyta, Haslea, Hemidinium, Hemitoma, Heribaudiella, Heteromastix, Heterothrix, Hibberdia, Hildenbrandia, Hillea, Holopedium, Homoeothrix, Hormanthonema, Hormotila, Hyalobrachion, Hyalocardium, Hyalodiscus, Hyalogonium, Hyalotheca, Hydrianum, Hydrococcus, Hydrocoleum, Hydrocoryne, Hydrodictyon, Hydrosera, Hydrurus, Hyella, Hymenomonas, Isthmochloron, Johannesbaptistia, Juranyiella, Karayevia, Kathablepharis, Katodinium, Kephyrion, Keratococcus, Kirchneriella, Klebsormidium, Kolbesia, Koliella, Komarekia, Korshikoviella, Kraskella, Lagerheimia, Lagynion, Lamprothamnium, Lemanea, Lepocinclis, Leptosira, Lobococcus, Lobocystis, Lobomonas, Luticola, Lyngbya, Malleochloris, Mallomonas, Mantoniella, Marssoniella, Martyana, Mastigocoleus, Gastogloia, Melosira, Merismopedia, Mesostigma, Mesotaenium, Micractinium, Micrasterias, Microchaete, Microcoleus, Microcystis, Microglena, Micromonas, Microspora, Microthamnion, Mischococcus, Monochrysis, Monodus, Monomastix, Monoraphidium, Monostroma, Mougeotia, Mougeotiopsis, Myochloris, Myromecia, Myxosarcina, Naegeliella, Nannochloris, Nautococcus, Navicula, Neglectella, Neidium, Nephroclamys, Nephrocytium, Nephrodiella, Nephroselmis, Netrium, Nitella, Nitellopsis, Nitzschia, Nodularia, Nostoc, Ochromonas, Oedogonium, Oligochaetophora, Onychonema, Oocardium, Oocystis, Opephora, Ophiocytium, Orthoseira, Oscillatoria, Oxyneis, Pachycladella, Palmella, Palmodictyon, Pnadorina, Pannus, Paralia, Pascherina, Paulschulzia, Pediastrum, Pedinella, Pedinomonas, Pedinopera, Pelagodictyon, Penium, Peranema, Peridiniopsis, Peridinium, Peronia, Petroneis, Phacotus, Phacus, Phaeaster, Phaeodermatium, Phaeophyta, Phaeosphaera, Phaeothamnion, Phormidium, Phycopeltis, Phyllariochloris, Phyllocardium, Phyllomitas, Pinnularia, Pitophora, Placoneis, Planctonema, Planktosphaeria, Planothidium, Plectonema, Pleodorina, Pleurastrum, Pleurocapsa, Pleurocladia, Pleurodiscus, Pleurosigma, Pleurosira, Pleurotaenium, Pocillomonas, Podohedra, Polyblepharides, Polychaetophora, Polyedriella, Polyedriopsis, Polygoniochloris, Polyepidomonas, Polytaenia, Polytoma, Polytomella, Porphyridium, Posteriochromonas, Prasinochloris, Prasinocladus, Prasinophyta, Prasiola, Prochlorphyta, Prochlorothrix, Protoderma, Protosiphon, Provasoliella, Prymnesium, Psammodictyon, Psammothidium, Pseudanabaena, Pseudenoclonium, Psuedocarteria, Pseudochate, Pseudocharacium, Pseudococcomyxa, Pseudodictyosphaerium, Pseudokephyrion, Pseudoncobyrsa, Pseudoquadrigula, Pseudosphaerocystis, Pseudostaurastrum, Pseudostaurosira, Pseudotetrastrum, Pteromonas, Punctastruata, Pyramichlamys, Pyramimonas, Pyrrophyta, Quadrichloris, Quadricoccus, Quadrigula, Radiococcus, Radiofilum, Raphidiopsis, Raphidocelis, Raphidonema, Raphidophyta, Peimeria, Rhabdoderma, Rhabdomonas, Rhizoclonium, Rhodomonas, Rhodophyta, Rhoicosphenia, Rhopalodia, Rivularia, Rosenvingiella, Rossithidium, Roya, Scenedesmus, Scherffelia, Schizochlamydella, Schizochlamys, Schizomeris, Schizothrix, Schroederia, Scolioneis, Scotiella, Scotiellopsis, Scourfieldia, Scytonema, Selenastrum, Selenochloris, Sellaphora, Semiorbis, Siderocelis, Diderocystopsis, Dimonsenia, Siphononema, Sirocladium, Sirogonium, Skeletonema, Sorastrum, Spermatozopsis, Sphaerellocystis, Sphaerellopsis, Sphaerodinium, Sphaeroplea, Sphaerozosma, Spiniferomonas, Spirogyra, Spirotaenia, Spirulina, Spondylomorum, Spondylosium, Sporotetras, Spumella, Staurastrum, Stauerodesmus, Stauroneis, Staurosira, Staurosirella, Stenopterobia, Stephanocostis, Stephanodiscus, Stephanoporos, Stephanosphaera, Stichococcus, Stichogloea, Stigeoclonium, Stigonema, Stipitococcus, Stokesiella, Strombomonas, Stylochrysalis, Stylodinium, Styloyxis, Stylosphaeridium, Surirella, Sykidion, Symploca, Synechococcus, Synechocystis, Synedra, Synochromonas, Synura, Tabellaria, Tabularia, Teilingia, Temnogametum, Tetmemorus, Tetrachlorella, Tetracyclus, Tetradesmus, Tetraedriella, Tetraedron, Tetraselmis, Tetraspora, Tetrastrum, Thalassiosira, Thamniochaete, Thorakochloris, Thorea, Tolypella, Tolypothrix, Trachelomonas, Trachydiscus, Trebouxia, Trentepholia, Treubaria, Tribonema, Trichodesmium, Trichodiscus, Trochiscia, Tryblionella, Ulothrix, Uroglena, Uronema, Urosolenia, Urospora, Uva, Vacuolaria, Vaucheria, Volvox, Volvulina, Westella, Woloszynskia, Xanthidium, Xanthophyta, Xenococcus, Zygnema, Zygnemopsis, and Zygonium. Additional cyanobacteria include members of the genus Chamaesiphon, Chroococcus, Cyanobacterium, Cyanobium, Cyanothece, Dactylococcopsis, Gloeobacter, Gloeocapsa, Gloeothece, Microcystis, Prochlorococcus, Prochloron, Synechococcus, Synechocystis, Cyanocystis, Dermocarpella, Stanieria, Xenococcus, Chroococcidiopsis, Myxosarcina, Arthrospira, Borzia, Crinalium, Geitlerinemia, Leptolyngbya, Limnothrix, Lyngbya, Microcoleus, Oscillatoria, Planktothrix, Prochiorothrix, Pseudanabaena, Spirulina, Starria, Symploca, Trichodesmium, Tychonema, Anabaena, Anabaenopsis, Aphanizomenon, Cyanospira, Cylindrospermopsis, Cylindrospermum, Nodularia, Nostoc, Scylonema, Calothrix, Rivularia, Tolypothrix, Chlorogloeopsis, Fischerella, Geitieria, Iyengariella, Nostochopsis, Stigonema and Thermosynechococcus.

Green non-sulfur bacteria include but are not limited to the following genera:

Chloroflexus, Chloronema, Oscillochloris, Heliothrix, Herpetosiphon, Roseiflexus, and Thermomicrobium.

Green sulfur bacteria include but are not limited to the following genera:

Chlorobium, Clathrochloris, and Prosthecochloris.

Purple sulfur bacteria include but are not limited to the following genera:

Allochromatium, Chromatium, Halochromatium, Isochromatium, Marichromatium, Rhodovulum, Thermochromatium, Thiocapsa, Thiorhodococcus, and Thiocystis,

Purple non-sulfur bacteria include but are not limited to the following genera: Phaeospirillum, Rhodobaca, Rhodobacter, Rhodomicrobium, Rhodopila, Rhodopseudomonas, Rhodothalassium, Rhodospirillum, Rodovibrio, and Roseospira.

Aerobic chemolithotrophic bacteria include but are not limited to nitrifying bacteria such as Nitrobacteraceae sp., Nitrobacter sp., Nitrospina sp., Nitrococcus sp., Nitrospira sp., Nitrosomonas sp., Nitrosococcus sp., Nitrosospira sp., Nitrosolobus sp., Nitrosovibrio sp.; colorless sulfur bacteria such as, Thiovulum sp., Thiobacillus sp., Thiomicrospira sp., Thiosphaera sp., Thermothrix sp.; obligately chemolithotrophic hydrogen bacteria such as Hydrogenobacter sp., iron and manganese-oxidizing and/or depositing bacteria such as Siderococcus sp., and magnetotactic bacteria such as Aquaspirillum sp.

Archaeobacteria include but are not limited to methanogenic archaeobacteria such as Methanobacterium sp., Methanobrevibacter sp., Methanothermus sp., Methanococcus sp., Methanomicrobium sp., Methanospirillum sp., Methanogenium sp., Methanosarcina sp., Methanolobus sp., Methanothrix sp., Methanococcoides sp., Methanoplanus sp.; extremely thermophilic S-Metabolizers such as Thermoproteus sp., Pyrodictium sp., Sulfolobus sp., Acidianus sp. and other microorganisms such as, Bacillus subtilis, Saccharomyces cerevisiae, Streptomyces sp., Ralstonia sp., Rhodococcus sp., Corynebacteria sp., Brevibacteria sp., Mycobacteria sp., and oleaginous yeast.

Preferred organisms for the manufacture of n-alkanes according to the methods discloused herein include: Arabidopsis thaliana, Panicum virgatum, Miscanthus giganteus, and Zea mays (plants); Botryococcus braunii, Chlamydomonas reinhardtii and Dunaliela salina (algae); Synechococcus sp PCC 7002, Synechococcus sp. PCC 7942, Synechocystis sp. PCC 6803, Thermosynechococcus elongatus BP-1 (cyanobacteria); Chlorobium tepidum (green sulfur bacteria), Chloroflexus auranticus (green non-sulfur bacteria); Chromatium tepidum and Chromatium vinosum (purple sulfur bacteria); Rhodospirillum rubrum, Rhodobacter capsulatus, and Rhodopseudomonas palusris (purple non-sulfur bacteria).

Yet other suitable organisms include synthetic cells or cells produced by synthetic genomes as described in Venter et al. US Pat. Pub. No. 2007/0264688, and cell-like systems or synthetic cells as described in Glass et al. US Pat. Pub. No. 2007/0269862.

Still, other suitable organisms include microorganisms that can be engineered to fix carbon dioxide bacteria such as Escherichia coli, Acetobacter aceti, Bacillus subtilis, yeast and fungi such as Clostridium ljungdahlii, Clostridium thermocellum, Penicillium chrysogenum, Pichia pastoris, Saccharomyces cerevisiae, Schizosaccharomyces pombe, Pseudomonas fluorescens, or Zymomonas mobilis.

A suitable organism for selecting or engineering is capable of autotrophic fixation of CO₂ to products. This would cover photosynthesis and methanogenesis. Acetogenesis, encompassing the three types of CO₂ fixation; Calvin cycle, acetyl-CoA pathway and reductive TCA pathway is also covered. The capability to use carbon dioxide as the sole source of cell carbon (autotrophy) is found in almost all major groups ofprokaryotes. The CO₂ fixation pathways differ between groups, and there is no clear distribution pattern of the four presently-known autotrophic pathways. See, e.g., Fuchs, G. 1989. Alternative pathways of autotrophic CO₂ fixation, p. 365-382. In H. G. Schlegel, and B. Bowien (ed.), Autotrophic bacteria. Springer-Verlag, Berlin, Germany. The reductive pentose phosphate cycle (Calvin-Bassham-Benson cycle) represents the CO₂ fixation pathway in almost all aerobic autotrophic bacteria, for example, the cyanobacteria.

For producing carbon-based compounds of interest via the recombinant expression of enzymes to enhance production of pyruvate or acetyl-CoA, an engineered cyanobacteria, e.g., a Synechococcus or Thermosynechococcus species, is preferred. Other preferred organisms include Synechocystis, Klebsiella oxytoca, Escherichia coli or Saccharomyces cerevisiae. Other prokaryotic, archaea and eukaryotic host cells are also encompassed within the scope of the present invention.

### Carbon-Based Compounds of Interest: Hydrocarbons & Alcohols

In various embodiments of the invention, desired hydrocarbons and/or alcohols of certain chain length or a mixture thereof can be produced. In certain aspects, the host cell produces at least one of the following carbon-based compounds of interest: 1-dodecanol, 1-tetradecanol, 1-pentadecanol, n-tridecane, n-tetradecane, 15:1 n-pentadecane, n-pentadecane, 16:1 n-hexadecene, n-hexadecane, 17:1 n-heptadecene, n-heptadecane, 16:1 n-hexadecen-ol, n-hexadecan-1-ol and n-octadecen-1-ol, as shown in the Examples herein. In other aspects, the carbon chain length ranges from C₁₀ to C₂₀. Accordingly, the invention provides production of various chain lengths of alkanes, alkenes and alkanols suitable for use as fuels & chemicals.

In preferred aspects, the methods provide culturing host cells for direct product secretion for easy recovery without the need to extract biomass. These carbon-based compounds of interest are secreted directly into the medium. Since the invention enables production of various defined chain length of hydrocarbons and alcohols, the secreted products are easily recovered or separated. The products of the invention, therefore, can be used directly or used with minimal processing.

### Fuel Compositions

In various embodiments, compositions produced by the methods of the invention are used as fuels. Such fuels comply with ASTM standards, for instance, standard specifications for diesel fuel oils D 975-09b, and Jet A, Jet A-1 and Jet B as specified in ASTM Specification D. 1655-68. Fuel compositions may require blending of several products to produce a uniform product. The blending process is relatively straightforward, but the determination of the amount of each component to include in a blend is much more difficult. Fuel compositions may, therefore, include aromatic and/or branched hydrocarbons, for instance, 75% saturated and 25% aromatic, wherein some of the saturated hydrocarbons are branched and some are cyclic. Preferably, the methods of the invention produce an array of hydrocarbons, such as C₁₃-C₁₇ or C₁₀-C₁₅ to alter cloud point. Furthermore, the compositions may comprise fuel additives, which are used to enhance the performance of a fuel or engine. For example, fuel additives can be used to alter the freezing/gelling point, cloud point, lubricity, viscosity, oxidative stability, ignition quality, octane level, and flash point. Fuels compositions may also comprise, among others, antioxidants, static dissipater, corrosion inhibitor, icing inhibitor, biocide, metal deactivator and thermal stability improver.

In addition to many environmental advantages of the invention such as CO₂ conversion and renewable source, other advantages of the fuel compositions disclosed herein include low sulfur content, low emissions, being free or substantially free of alcohol and having high cetane number.

Alternative Recombinant Methods of Biosynthesis of Pyruvate or Acetyl-CoA

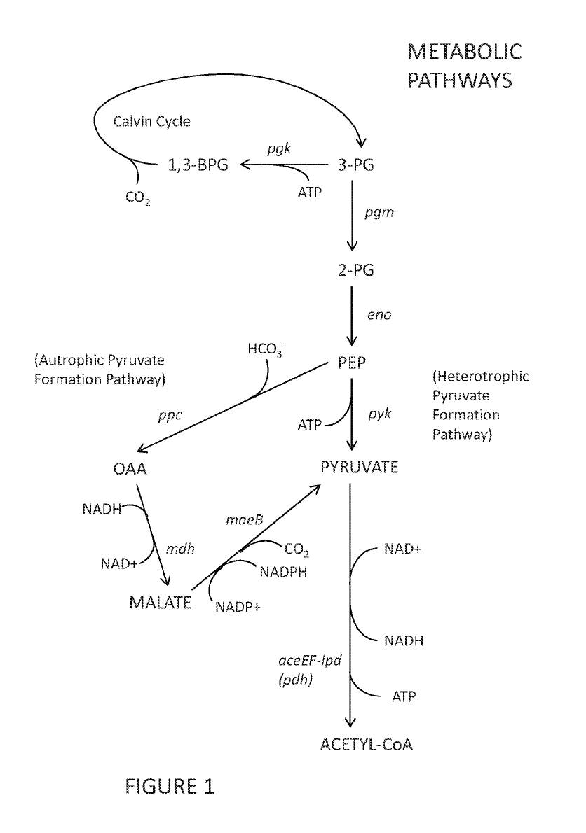

Generally the desired end products in organisms engineered to have high flux to pyruvate are products other than pyruvate itself. These end products can be divided into two types: 1) non-acetyl-CoA derivatives of pyruvate (in which decreased expression of pyruvate dehydrogenase increases pyruvate available for non-acetyl-CoA derivatives, but also diminishes available NADH) and 2) derivatives of acetyl-CoA (in which expression of pyruvate dehydrogenase or an alternative is necessary to form acetyl-CoA). Methods for optimizing yields of these two types of products are considered separately. FIG. 1 shows pathways around the pyruvate node in Synechococcus elongatus PCC 7002, an exemplary photosynthetic microbe. Endogenous enzymes/genes are shown.

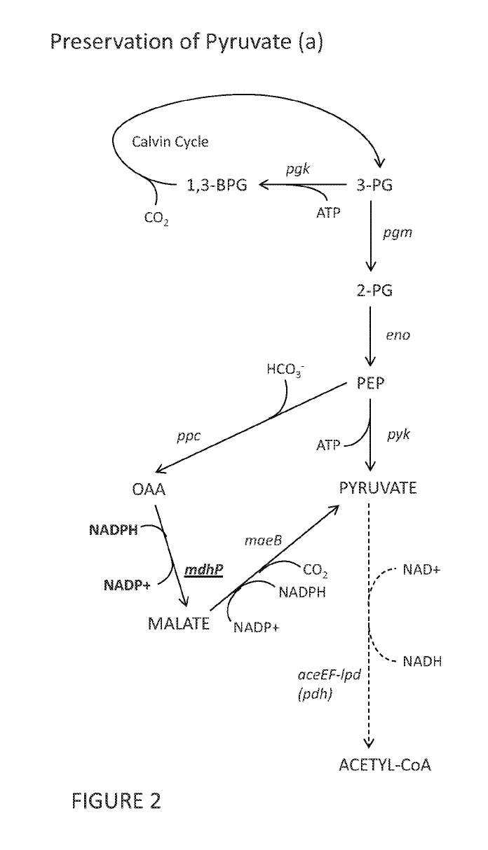

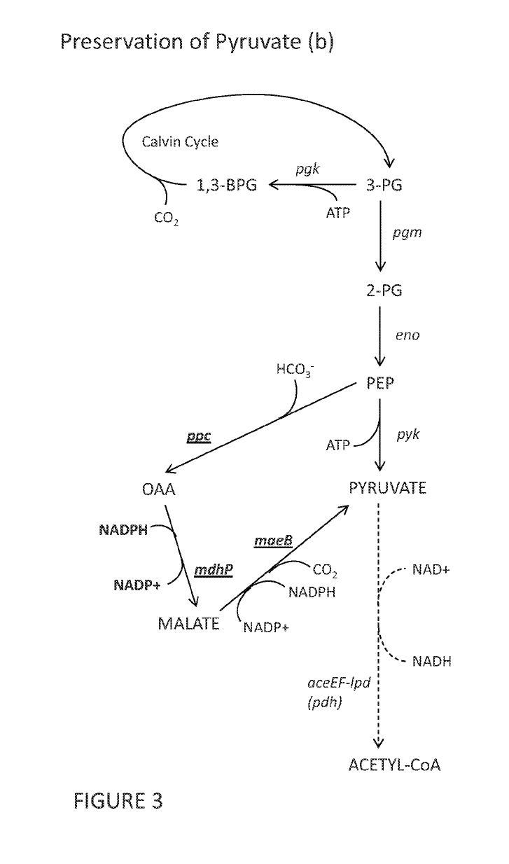

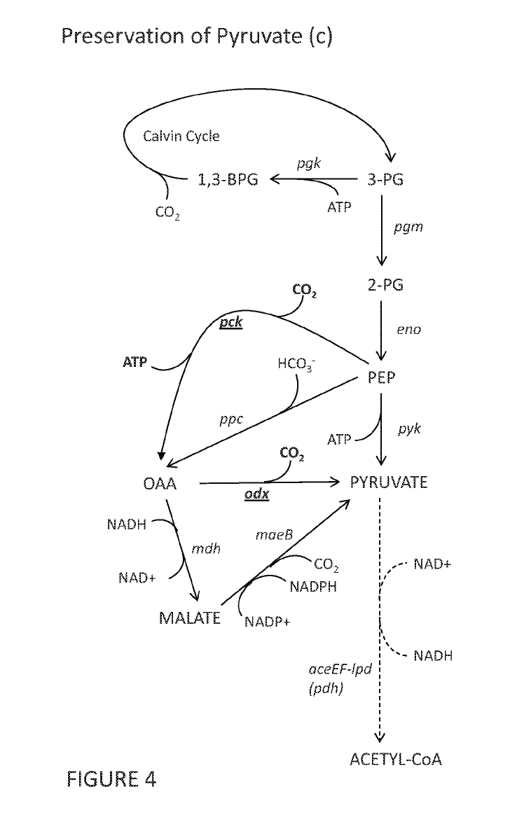

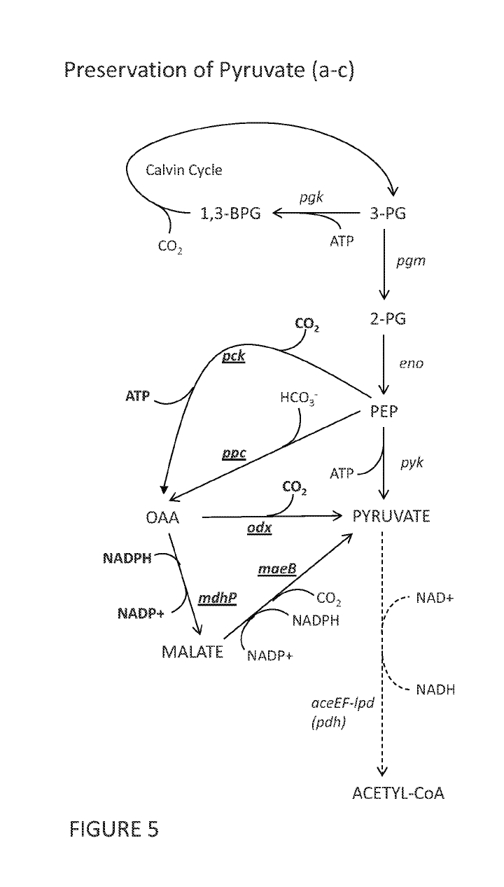

In one embodiment for optimizing yields of non-acetyl-CoA derivatives of pyruvate, the cofactor dependence of Mdh (EC 1.1.1.82) is changed from NADH to NADPH (FIG. 2). In another embodiment, recombinant maeB is introduced or endogenous maeB expression is upregulated in the host cell to increase flux to pyruvate (EC 1.1.1.38) (FIG. 3). In still another embodiment, recombinant ppc is introduced or endogenous ppc expression is upregulated in the host cell to increase biosynthesis of oxaloacetic acid from phosphoenol pyruvate (EC 4.1.1.31) to increase flux to pyruvate (FIG. 3). In yet another embodiment, recombinant odx is introduced into the host cell to allow biosynthesis of pyruvate from oxaloacetic acid (EC 4.1.1.3) without consumption of NADH (FIG. 4). In another embodiment, recombinant pck is introduced into the host cell to enhance the rate of biosynthesis of oxaloacetic acid from phosphoenol pyruvate (EC 4.1.1.49), which increases flux to pyruvate (FIG. 4). Optimized combinations of any of the described embodiments may also be used to enhance flux to pyruvate (i.e., increase the rate of biosynthesis of pyruvate in the host cell) (FIG. 5). For example, in one embodiment, pck is expressed in combination with mdhP to increase the rate of formation of oxaloacetic acid and to generate malate (a precursor to pyruvate) without consumption of NADH.

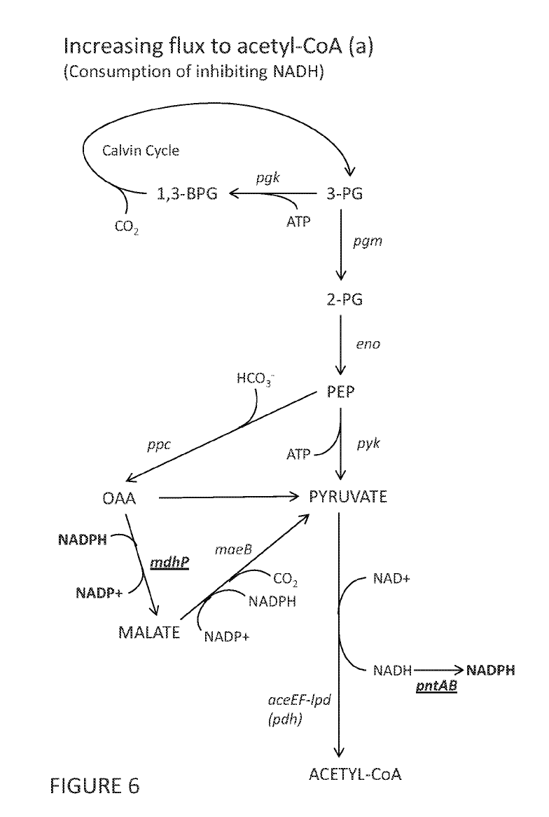

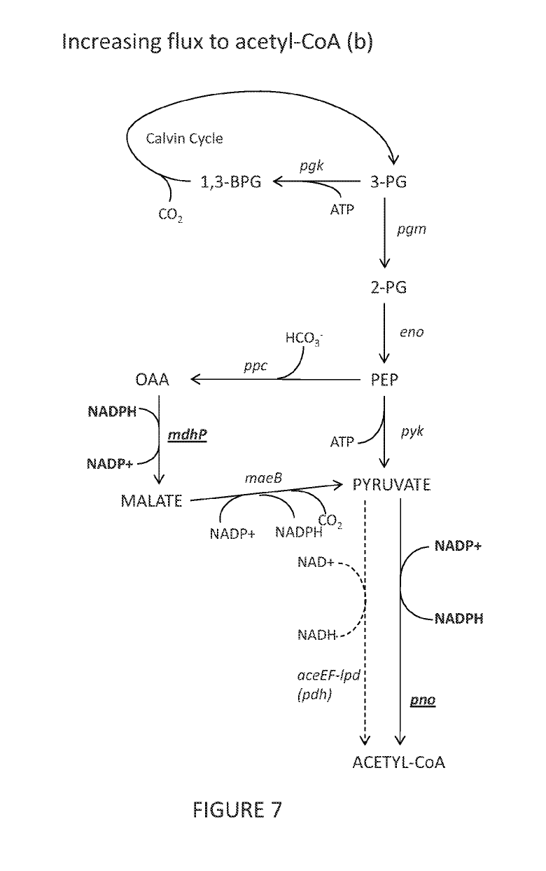

In one embodiment for increasing flux to acetyl-CoA, the cofactor dependence of the pyruvate dehydrogenase complex is switched from NAD+ to NADP+. In another embodiment, increasing flux to acetyl-CoA can be achieved, e.g., by employing a recombinant transhydrogenase (e.g., from EC 1.6.1.2) to convert the excess NADH to NADPH, (e.g., as shown in FIG. 6), by finding alternative pathways from pyruvate to acetyl-CoA and incorporating recombinant enzymes for the biosynthesis of acetyl-CoA (e.g., via EC 1.2.1.51, via EC 1.2.7.-, or via EC 4.1.1.1, EC 1.2.1.4, and EC 6.2.1.1, in combination) (e.g., as shown in FIGS. 7-9).

In another embodiment, the methods and compositions described above are practiced in a heterotrophic organism in place of a cyanobacterium.

The following examples are for illustrative purposes and are not intended to limit the scope of the present invention.

### EXAMPLE 1

An alternative pathway for the enzymatic synthesis of pyruvate. An enzymatic process for the native production of pyruvate in Synechococcus elongatus PCC 7002 is shown in FIG. 1. To increase available pyruvate in a host cell for downstream biosynthesis of carbon-based products of interest, we introduce at least one recombinant gene into a host cell, e.g., Synechococcus elongatus PCC 7002 using standard molecular biology techniques, as discussed above in the detailed description. The recombinant gene or genes will express an enzyme to allow biosynthesis of pyruvate without requiring NADH cofactor.

In one embodiment, we will recombinantly introduce and express an mdh gene (such as from Pisum sativum) to consume NADPH instead of NADH (“mdhP”, SEQ ID NO: 1) (see, e.g., Reng, W., et al., (1993). Eur. J. Biochem. 217:189-197) in a host cell (FIG. 2). This may or may not be accompanied by knockout of the native mdh gene. In another embodiment, we remove the targeting signal from MdhP, and modify MdhP by altering the amino acid sequence such that the enzyme is equally active in the reduced and oxidized state (SEQ ID NO: 2). This involves the four mutations C23A, C28A, C206A, and C376A. See sequence below (mature protein with the four mutations indicated). This modified MdhP is recombinantly introduced into and expressed in a host cell.

In another embodiment, we will express ppc (SEQ ID NO: 3), mdhP (P. sativum) (SEQ ID NO: 1 or SEQ ID NO: 2), and maeB (SEQ ID NO: 5) in the host cell (FIG. 3). ppc and maeB may be native to the host or recombinantly imported into the host from another host. MaeB will generate NADPH and not NADH, as is the case with the enzyme from Synechococcus elongatus PCC 7002. In one aspect, we will introduce the S8D mutant of Sorghum Ppc (SEQ ID NO: 4), which is more active and less inhibited by malate than the wild-type Sorghum enzyme (see, e.g., Chollet, R. (1996). Annu. Rev. Plant Physiol. Plant Mol. Biol. 47:273-98.). In another embodiment, we will express the S8D mutant of Sorghum Ppc (SEQ ID NO: 4), mdhP (P. sativum) (SEQ ID NO: 1 or SEQ ID NO: 2), and maeB (SEQ ID NO: 5) in the host cell.

In another embodiment, we will recombinantly introduce and express odx (SEQ ID NO: 6) as a short-circuit of all cofactor dependence (FIG. 4). In another embodiment, we will also enhance production of ppc (SEQ ID NO: 3). In still another embodiment, we will recombinantly introduce and express pck (such as from E. coli, SEQ ID NO: 7) to capture an additional ATP. (FIG. 4). odx can be obtained from Corynebacterium glutamicum (SEQ ID NO: 6) (see, e.g., Klaffl, S. and B. J. Eikmanns, (2010). J. Bacteriol. 192:2604-12). Any of these strategies can be employed instead in a strain attenuated for mdh activity to preserve oxaloacetate for Odx.

Two or more of the embodiments described in this example may be employed in combination to generate excess pyruvate in the host cell (FIG. 5). In addition, one or more of the above embodiments may be performed in conjunction with attenuation of native pyruvate dehydrogenase activity to increase pyruvate available for conversion to non-acetyl-CoA derived carbon-based compounds of interest.

Additional recombinant genes may be present in and/or introduced to the host cell depending on the desired product (i.e., carbon-based compound of interest). For example, pdc and an alcohol dehydrogenase gene will be overexpressed in conjunction with any of the embodiments in Example 1 for the biosynthesis of ethanol from pyruvate.

### EXAMPLE 2

An alternative pathway for the enzymatic synthesis of acetyl-CoA. An enzymatic process for the native production of acetyl-CoA in Synechococcus elongatus PCC 7002 is shown in FIG. 1. To increase available acetyl-CoA in a host cell for downstream biosynthesis of carbon-based products of interest, we introduce at least one recombinant gene into a host cell, e.g., Synechococcus elongatus PCC 7002 using standard molecular biology techniques, as discussed above in the detailed description. The recombinant gene or genes will express an enzyme to allow biosynthesis of acetyl-CoA without generating excess NADH in the host cell.

In one embodiment, we will recombinantly introduce and express an NADPH-producing transhydrogenase system in a host cell, e.g., pntAB from Escherichia coli (SEQ ID NO: 8 and SEQ ID NO: 9) (FIG. 6) (see, e.g., Sauer, U., F. et al. (2004). J. Biol. Chem. 279:6613-19). In one embodiment, NADPH-dependent mdhP (SEQ ID NO: 1 or SEQ ID NO: 2) is also expressed in the host cell.

In another embodiment, we will recombinantly introduce and express NADPH-generating pyruvate dehydrogenase called pno from Euglena gracilis (SEQ ID NO: 10) or Cryptosporidium parvum (SEQ ID NO: 11), in place of or in conjunction with native pyruvate dehydrogenase (pdh) (FIG. 7). In one embodiment, NADPH-dependent mdhP (SEQ ID NO: 1 or SEQ ID NO: 2) is also expressed in the host cell.

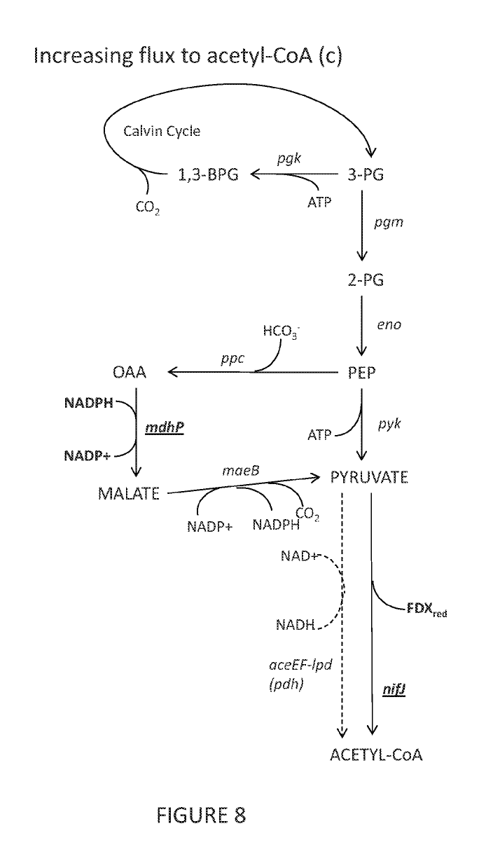

In another embodiment, we will overexpress or recombinantly introduce and express pyruvate:ferredoxin oxidoreductase (nifJ) (SEQ ID NO: 12) (FIG. 8). The reduced ferredoxin produced can be converted to NADPH by native systems such as PetH. In one embodiment, NADPH-dependent mdhP (SEQ ID NO: 1 or SEQ ID NO: 2) is also expressed in the host cell.

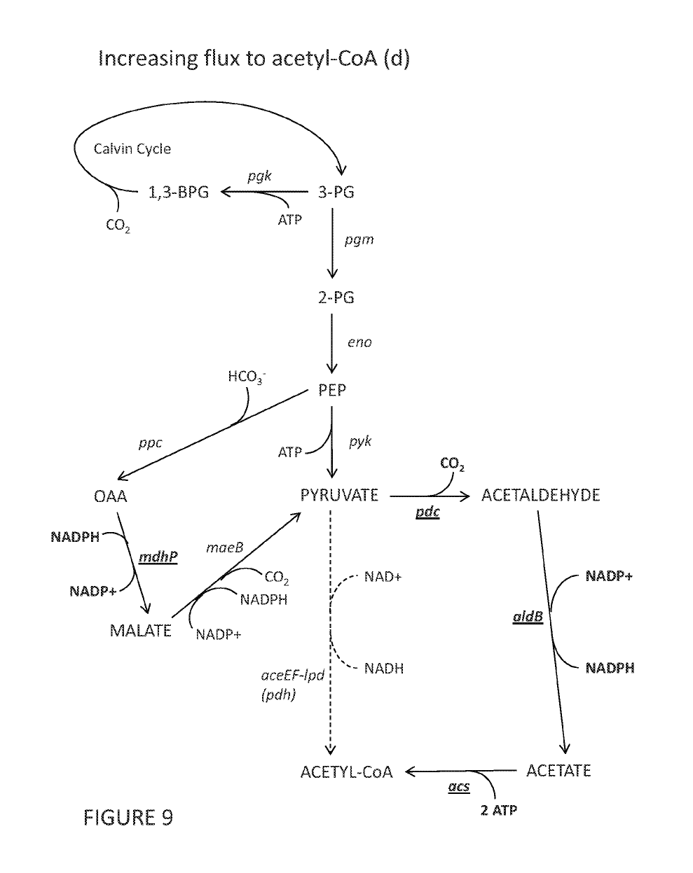

In another embodiment, we will recombinantly introduce and express a three-part reconstituted NADPH-generating pyruvate dehydrogenase, assembled from pyruvate decarboxylase such as pdc from Zymomonas mobilis (SEQ ID NO: 13), NADP-dependent acetaldehyde dehydrogenase such as aldB from E. coli (SEQ ID NO: 14), and acetyl-CoA synthetase such as acs from E. coli (SEQ ID NO: 15) (FIG. 9). In one embodiment, NADPH-dependent mdhP (SEQ ID NO: 1 or SEQ ID NO: 2) is also expressed in the host cell.

Two or more of the embodiments described in this Example may be employed in combination to generate excess pyruvate in the host cell. In addition, one or more of the embodiments described in this Example may be performed in conjunction with attenuation of native pyruvate dehydrogenase activity to mitigate excess NADH production.

Additional recombinant genes may be present in and/or introduced to the host cell depending on the desired product (i.e., carbon-based compound of interest).

Additional information regarding the enzymes used in the pathways described above can be found in Table 1.

TABLE 1
Pathway Enzyme Information

| Enzyme | Organism | EC number | SEQ ID NO: | locus/loci* | GenBank Acc. No. |
|---|---|---|---|---|---|
| MdhP | Pisum sativum | 1.1.1.82 | SEQ ID NO: 1 | — | CAA52614 |
| MdhP | Pisum sativum | 1.1.1.82 | SEQ ID NO: 2 | — | — |
| (modified) | | | | | |
| Ppc | Synechococcus | 4.1.1.31 | SEQ ID NO: 3 | SYNPCC7002_A1414 | ACA99405 |
| | elongatus PCC 7002 | | | | |
| Ppc_S8D | Sorghum bicolor | 4.1.1.31 | SEQ ID NO: 4 | — | CAPP3_SORBI^{†} |
| MaeB | Synechococcus | 1.1.1.38 | SEQ ID NO: 5 | SYNPCC7002_A0448 | ACA98456 |
| | elongatus PCC 7002 | | | | |
| Odx | Corynebacterium | 4.1.1.3 | SEQ ID NO: 6 | Cg1458 | NC_006958 |
| | glutamicum | | | | |
| Pck | Escherichia coli | 4.1.1.49 | SEQ ID NO: 7 | b3403 | NP_417862 |
| PntAB | Escherichia coli | 1.6.1.2 | SEQ ID NO: 8 | b1603, b1602 | NP_416120, |
| | | | SEQ ID NO: 9 | | NP_416119 |
| Pno | Euglena gracilis | 1.2.1.51 | SEQ ID NO: 10 | — | CAC37628 |
| Pno | Cryptosporidium | 1.2.1.51 | SEQ ID NO: 11 | CMU_012830 | XM_002140922 |
| | muris RN66 | | | | |
| NifJ | Synechococcus | 1.2.7.— | SEQ ID NO: 12 | SYNPCC7002_A1443 | ACA99434 |
| | elongatus PCC 7002 | | | | |
| Pdc | Zymomonas | 4.1.1.1 | SEQ ID NO: 13 | ZMO1360 | AAV89984 |
| | mobilis ZM4 | | | | |
| AldB | Escherichia coli | 1.2.1.4 | SEQ ID NO: 14 | b3588 | NP_418045 |
| Acs | Escherichia coli | 6.2.1.1 | SEQ ID NO: 15 MM | b4069 | NP_418493 |

*when from a sequenced genome
^{†}CAPP3_SORBI is the native sequence; shown below it has the S8D mutation

A number of embodiments of the invention have been described. Nevertheless, it will be understood that various modifications may be made without departing from the spirit and scope of the invention. All publications, patents and other references mentioned herein are hereby incorporated by reference in their entirety.

Informal Sequence Listing

SEQ ID NO: 1
Pisum sativum MdhP enzyme (EC 1.1.1.82) amino acid sequence
MALTQLNSTCSKPQLHSSSQLSFLSRTRTRTLPRHYHSTFAPLHRTQHARISCSVAPNQVQVPAAQTQDPKGKPD
CYGVFCLTYDLKAEEETKSWKKLINIAVSGAAGMISNHLLFKLASGEVFGPDQPIALKLLGSERSIQALEGVAME
LEDSLFPLLREVVISIDPYEVFQDAEWALLIGAKPRGPGVERAALLDINGQIFAEQGKALNAVASRNAKVIVVGN
PCNTNALICLKNAPNIPAKNFHALTRLDENRAKCQLALKAGVFYDKVSNMTIWGNHSTTQVPDFLNARIDGLPVK
EVIKDNKWLEEEFTEKVQKRGGVLIQKWGRSSAASTSVSIVDAIRSLITPTPEGDWFSSGVYTNGNPYGIAEDIV
FSMPCRSKGDGDYELVNDVIFDDYLRQKLAKTEAELLAEKKCVAHLTGEGIAVCDLPGDTMLPGEM
SEQ ID NO: 2
Pisum sativum MdhP enzyme (modified) (EC 1.1.1.82) amino
acid sequence
(mature MdhP protein, minus chloroplast targeting signal,
including the four C → A mutations)
MSVAPNQVQVPAAQTQDPKGKPDAYGVFALTYDLKAEEETKSWKKLINIAVSGAAGMISNHLLFKLASGEVFGPD
QPIALKLLGSERSIQALEGVAMELEDSLFPLLREVVISIDPYEVFQDAEWALLIGAKPRGPGVERAALLDINGQI
FAEQGKALNAVASRNAKVIVVGNPCNTNALICLKNAPNIPAKNFHALTRLDENRAKAQLALKAGVFYDKVSNMTI
WGNHSTTQVPDFLNARIDGLPVKEVIKDNKWLEEEFTEKVQKRGGVLIQKWGRSSAASTSVSIVDAIRSLITPTP
EGDWFSSGVYTNGNPYGIAEDIVFSMPCRSKGDGDYELVNDVIFDDYLRQKLAKTEAELLAEKKCVAHLTGEGIA
VADLPGDTMLPGEM
SEQ ID NO: 3
Synechococcus elongatus PCC 7002 phosphoenol pyruvate
carboxylase (Ppc) amino acid sequence
MNQVMHPPSAEAELLSTSQSLLRQRLTLVEDIWQAVLQKECGQKLVERLNHLRATRTADGQSLNFSPSSISELIE
TLDLEDAIRAARAFALYFQLINSVEQHYEQREQQQFRRNLASANASEANGNSVHTEIAPTQAGTFDWLFPHLKHQ
NMPPQTIQRLLNQLDIRLVFTAHPTEIVRHTIRNKQRRIAGILRQLDQTEEGLKSLGTSDSWEIENIQQQLTEEI
RLWWRTDELHQFKPQVLDEVDYALHYFEEVLFDTLPELSVRLQQALKASEPTLKVPTTNFCNEGSWVGGDRDGNP
SVTPDVTWKTACYQRGLVLERYIASVESLSDVLSLSLHWSNVLPDLLDSLEQDQNIFPDIYETLAIRYRQEPYRL
KLAYIKRRLENTLERNRRLANMPAWENKVEAADDKVYICGQEFLADLKLIRESLVQTEINCAALDKLICQVEIFS
FVLTRLDFRQESTRHSDAIAEIVDYLGVLPKSYNDLSDAEKTTWLVQELKTRRPLIPKEMHFSERTVETIQTLQV
LRRLQQEFGIGICQTYIISMTNEVSDVLEVLLLAQEAGLYDPLTGMTTIRIAPLFETVDDLRNAPEIMQALFEIP
LYRACLAGGYEPPADGRCDETFGDRLVPNLQEIMLGYSDSNKDSGFLSSNWEIHKAQKNLQQVADPYGIDLRIFH
GRGGSVGRGGGPAYAAILAQPPNTINGRIKITEQGEVLASKYSLPDLALYHLESVSTAVIQSSLLASGFDDIQPW
NRIMEDLSQRSRAAYRALIYEEPDFLDFFMSVTPIPEISQLQISSRPARRKKGNKDLSSLRAIPWVFSWTQSRFL
VPAWYGVGTALQGFFEEDPVENLKLMRYFYSKWPFFRMVISKVEMTLSKVDLQMASHYVHELAEKEDIPRFEKLL
EQISQEYNLTKRLILEITENEALLDGDRPLQRSVQLRNGTIVPLGFLQVSLLKRLRQYTRETQASIVHFRYSKEE
LLRGALLTINGIAAGMRNTG
SEQ ID NO: 4
Sorghum bicolor phosphoenol pyruvate carboxylase S8D
(S8D mutation bold, underlined)
(Ppc S8D) amino acid sequence
MASERHHDIDAQLRALAPGKVSEELIQYDALLVDRFLDILQDLHGPSLREFVQECYEVSADYEGKKDTSKLGELG
AKLTGLAPADAILVASSILHMLNLANLAEEVELAHRRRNSKLKHGDFSDEGSATTESDIEETLKRLVSLGKTPAE
VFEALKNQSVDLVFTAHPTQSARRSLLQKNARIRNCLTQLSAKDVTVEDKKELDEALHREIQAAFRTDEIRRAQP
TPQDEMRYGMSYIHETVWNGVPKFLRRVDTALKNIGINERLPYDVPLIKFCSWMGGDRDGNPRVTPEVTRDVCLL
SRMMAANLYINQVEDLMFELSMWRCNDELRARAEEVQSTPASKKVTKYYIEFWKQIPPNEPYRVILGAVRDKLYN
TRERARHLLATGFSEISEDAVFTKIEEFLEPLELCYKSLCECGDKAIADGSLLDLLRQVFTFGLSLVKLDIRQES
ERQTDVIDAITTHLGIGSYRSWPEDKRMEWLVSELKGKRPLLPPDLPMTEEIADVIGAMRVLAELPIDSFGPYII
SMCTAPSDVLAVELLQRECGIRQTLPVVPLFERLADLQAAPASVEKLFSTDWYINHINGKQQVMVGYSDSGKDAG
RLSAAWQLYVAQEEMAKVAKKYGVKLTLFHGRGGTVGRGGGPTHLAILSQPPDTINGSIRVTVQGEVIEFMFGEE
NLCFQSLQRFTAATLEHGMHPPVSPKPEWRKLMEEMAVVATEEYRSVVVKEPRFVEYFRSATPETEYGKMNIGSR
PAKRRPGGGITTLRAIPWIFSWTQTRFHLPVWLGVGAAFKWAIDKDIKNFQKLKEMYNEWPFFRVTLDLLEMVFA
KGDPGIAGLYDELLVAEELKPFGKQLRDKYVETQQLLLQIAGHKDILEGDPYLKQGLRLRNPYITTLNVFQAYTL
KRIRDPSFKVTPQPPLSKEFADENKPAGLVKLNGERVPPGLEDTLILTMKGIAAGMQNTG
SEQ ID NO: 5
Synechococcus elongatus PCC 7002 NADPH-linked malic
enzyme (MaeB) amino acid sequence
MVALTPNPSYSVTIQLRIPNRAGTLANVTQAIANVGGSLGNIELVERDLKFLIRNITVDASSEKHAGDIVD
AVKAVPEIEILKVLDRTFAIHQGGKITVESRIPLTVQSDLAMAYTPGVGRVCQAIAEKPEQVYDLTVKSNM
VAIVTDGSAVLGLGNLGPEAAMPVMEGKAMLFKEFAGVNAFPICLATQDVEEIVRTVKYLAPVFGGVNLED
IAAPRCFEIEQRLKAETDIPIFHDDQHGTAIVSLAALFNALKLVKKDLQSVKIVINGAGAAGVAIAKLLRQ
AGATDIWMCDSKGIISSQRDNLTPEKRAFAVAASGTLAEAMQDADIFLGVSAPGVVTPAMVTSMAKDPIVF
AMANPIPEIQPELIMDKVAVVATGRSDYPNQINNVLAFPGVFRGALDCRASSITTNMCLQAAQAIASLVSP
AQLDREHIIPSVFDQRVAAAVAAAVGQAARQDGVARR
SEQ ID NO: 6
Corynebacterium glutamicum oxaloacetate decarboxylase
(Odx) amino acid sequence
MRFGRIATPDGMCFCSIEGEGDDVANLTAREIEGTPFTEPKFTGREWPLKDVRLLAPMLPSKVVAIGRNYADHVA
EVFKKSAESLPPTLFLKPPTAVTGPESPIRIPSFATKVEFEGELAVVIGKPCKNVKADDWKSVVLGFTIINDVSS
RDLQFADGQWARAKGIDTFGPIGPWIETDINSIDLDNLPIKARLTHDGETQLKQDSNSNQMIMKMGEIIEFITAS
MTLLPGDVIATGSPAGTEAMVDGDYIEIEIPGIGKLGNPVVDA
SEQ ID NO: 7
E. Coli phosphoenolpyruvate carboxykinase (Pck)
amino acid sequence
MRVNNGLTPQELEAYGISDVHDIVYNPSYDLLYQEELDPSLTGYERGVLTNLGAVAVDTGIFTGRSPKDKYIVRD
DTTRDTFWWADKGKGKNDNKPLSPETWQHLKGLVTRQLSGKRLFVVDAFCGANPDTRLSVRFITEVAWQAHFVKN
MFIRPSDEELAGFKPDFIVMNGAKCTNPQWKEQGLNSENFVAFNLTERMQLIGGTWYGGEMKKGMFSMMNYLLPL
KGIASMHCSANVGEKGDVAVFFGLSGTGKTTLSTDPKRRLIGDDEHGWDDDGVFNFEGGCYAKTIKLSKEAEPEI
YNAIRRDALLENVTVREDGTIDFDDGSKTENTRVSYPIYHIDNIVKPVSKAGHATKVIFLTADAFGVLPPVSRLT
ADQTQYHFLSGFTAKLAGTERGITEPTPTFSACFGAAFLSLHPTQYAEVLVKRMQAAGAQAYLVNTGWNGTGKRI
SIKDTRAIIDAILNGSLDNAETFTLPMFNLAIPTELPGVDTKILDPRNTYASPEQWQEKAETLAKLFIDNFDKYT
DTPAGAALVAAGPKL
SEQ ID NO: 8
E. coli transhydrogenase (PntA) amino acid sequence
MRIGIPRERLTNETRVAATPKTVEQLLKLGFTVAVESGAGQLASFDDKAFVQAGAEIVEGNSVWQSEIILKVNAP
LDDEIALLNPGTTLVSFIWPAQNPELMQKLAERNVTVMAMDSVPRISRAQSLDALSSMANIAGYRAIVEAAHEFG
RFFTGQITAAGKVPPAKVMVIGAGVAGLAAIGAANSLGAIVRAFDTRPEVKEQVQSMGAEFLELDFKEEAGSGDG
YAKVMSDAFIKAEMELFAAQAKEVDIIVTTALIPGKPAPKLITREMVDSMKAGSVIVDLAAQNGGNCEYTVPGEI
FTTENGVKVIGYTDLPGRLPTQSSQLYGTNLVNLLKLLCKEKDGNITVDFDDVVIRGVTVIRAGEITWPAPPIQV
SAQPQAAQKAAPEVKTEEKCTCSPWRKYALMALAIILFGWMASVAPKEFLGHFTVFALACVVGYYVVWNVSHALH
TPLMSVTNAISGIIVVGALLQIGQGGWVSFLSFIAVLIASINIFGGFTVTQRMLKMFRKN
SEQ ID NO: 9
E. coli transhydrogenase (PntB) amino acid sequence
MSGGLVTAAYIVAAILFIFSLAGLSKHETSRQGNNFGIAGMAIALIATIFGPDTGNVGWILLAMVIGGAIGIRLA
KKVEMTEMPELVAILHSFVGLAAVLVGFNSYLHHDAGMAPILVNIHLTEVFLGIFIGAVTFTGSVVAFGKLCGKI
SSKPLMLPNRHKMNLAALVVSFLLLIVFVRTDSVGLQVLALLIMTAIALVFGWHLVASIGGADMPVVVSMLNSYS
GWAAAAAGFMLSNDLLIVTGALVGSSGAILSYIMCKAMNRSFISVIAGGFGTDGSSTGDDQEVGEHREITAEETA
ELLKNSHSVIITPGYGMAVAQAQYPVAEITEKLRARGINVRFGIHPVAGRLPGHMNVLLAEAKVPYDIVLEMDEI
NDDFADTDTVLVIGANDTVNPAAQDDPKSPIAGMPVLEVWKAQNVIVFKRSMNTGYAGVQNPLFFKENTHMLFGD
AKASVDAILKAL
SEQ ID NO: 10
Euglena gracilis pyruvate dehydrogenase (Pno) amino
acid sequence
MKQSVRPIISNVLRKEVALYSTIIGQDKGKEPTGRTYTSGPKPASHIEVPHHVTVPATDRTPNPDAQFFQSVDGS
QATSHVAYALSDTAFIYPITPSSVMGELADVWMAQGRKNAFGQVVDVREMQSEAGAAGALHGALAAGAIATTFTA
SQGLLLMIPNMYKIAGELMPSVIHVAARELAGHALSIFGGHADVMAVRQTGWAMLCSHTVQQSHDMALISHVATL
KSSIPFVHFFDGFRTSHEVNKIKMLPYAELKKLVPPGTMEQHWARSLNPMHPTIRGTNQSADIYFQNMESANQYY
TDLAEVVQETMDEVAPYIGRHYKIFEYVGAPDAEEVTVLMGSGATTVNEAVDLLVKRGKKVGAVLVHLYRPWSTK
AFEKVLPKTVKRIAALDRCKEVTALGEPLYLDVSATLNLFPERQNVKVIGGRYGLGSKDFIPEHALAIYANLASE
NPIQRFTVGITDDVTGTSVPFVNERVDTLPEGTRQCVFWGIGSDGTVGANRSAVRIIGDNSDLMVQAYFQFDAFK
SGGVTSSHLRFGPKPITAQYLVTNADYIACHFQEYVKRFDMLDAIREGGTFVLNSRWTTEDMEKEIPADFRRNVA
QKKVRFYNVDARKICDSFGLGKRINMLMQACFFKLSGVLPLAEAQRLLNESIVHEYGKKGGKVVEMNQAVVNAVF
AGDLPQEVQVPAAWANAVDTSTRTPTGIEFVDKIMRPLMDFKGDQLPVSVMTPGGTFPVGTTQYAKRAIAAFIPQ
WIPANCTQCNYCSYVCPHATIRPFVLTDQEVQLAPESFVTRKAKGDYQGMNFRIQVAPEDCTGCQVCVETCPDDA
LEMTDAFTATPVQRTNWEFAIKVPNRGTMTDRYSLKGSQFQQPLLEFSGACEGCGETPYVKLLTQLFGERTVIAN
ATGCSSIWGGTAGLAPYTTNAKGQGPAWGNSLFEDNAEFGFGIAVANAQKRSRVRDCILQAVEKKVADEGLTTLL
AQWLQDWNTGDKTLKYQDQIIAGLAQQRSKDPLLEQIYGMKDMLPNISQWIIGGDGWANDIGFGGLDHVLASGQN
LNVLVLDTEMYSNTGGQASKSTHMASVAKFALGGKRTNKKNLTEMAMSYGNVYVATVSHGNMAQCVKAFVEAESY
DGPSLIVGYAPCIEHGLRAGMARMVQESEAAIATGYWPLYRFDPRLATEGKNPFQLDSKRIKGNLQEYLDRQNRY
VNLKKNNPKGADLLKSQMADNITARFNRYRRMLEGPNTKAAAPSGNHVTILYGSETGNSEGLAKELATDFERREY
SVAVQALDDIDVADLENMGFVVIAVSTCGQGQFPRNSQLFWRELQRDKPEGWLKNLKYTVFGLGDSTYYFYCHTA
KQIDARLAALGAQRVVPIGFGDDGDEDMFHTGFNNWIPSVWNELKTKTPEEALFTPSIAVQLTPNATPQDFHFAK
STPVLSITGAERITPADHTRNFVTIRWKTDLSYQVGDSLGVFPENTRSVVEEFLQYYGLNPKDVITIENKGSREL
PHCMAVGDLFTKVLDILGKPNNRFYKTLSYFAVDKAEKERLLKIAEMGPEYSNILSEMYHYADIFHMFPSARPTL
QYLIEMIPNIKPRYYSISSAPIHTPGEVHSLVLIDTWITLSGKHRTGLTCTMLEHLQAGQVVDGCIHPTAMEFPD
HEKPVVMCAMGSGLAPFVAFLRERSTLRKQGKKTGNMALYFGNRYEKTEFLMKEELKGHINDGLLTLRCAFSRDD
PKKKVYVQDLIKMDEKMMYDYLVVQKGSMYCCGSRSFIKPVQESLKHCFMKAGGLTAEQAENEVIDMFTTGRYNI
EAW
SEQ ID NO: 11
Cryptosporidium parvum pyruvate dehydrogenase (Pno)
amino acid sequence
MKSEIVDGCVAACHIAYACSEVAFIYPITPSSSISEAADSWMVKGKKNLFDQVVSVVEMQSEMGSAGALHGSLCV
GCVTTTFTASQGLLLMIPNMYKIAGELWPCVFHVTARALATSSLSIFGDHNDIMAARQTGWAFLGAMTVQEVMDL
ALVAHISTLESSMPFVHFFDGFRTSHELQKIEMIDYDTIKALYPYDKLRAFRSRALNPTHPVLRGTATSSDVYFQ
TVESRNAYYDAVPTIVQDVMNKVAKYTGRQYNLFDYYGYKEAEYVIVVMGSGGLTIEEMIEYLIKESNEKVGMIK
VRLFRPWSPDTFAKVLPTTVRRITVLERCKESGALGEPLYLDVSTTIMRIMQSDSRYKNISVIGGRYGLASKEFT
PGMALSIWENMRSESPIQNFSVGINDDVTFKSLQIRQPKLDLLTDETRQCMFWGLGSDGTVSANKNAIKIIGEST
NLFVQGYFAYDAKKAGGATMSHLRFGPKPIKSPYLLQRCDYIAVHHPSYIYKFDVLENIKENGIFVLNCSWKSVD
KISEELPARIKSIIARNNIRMYVVDAQDVAIRANLGRRINNILMVAFFRLANIIPFEEAINLIKDAIQKSYSKKG
EAVIKSNWRAVDLALESLIEVKYNRDAWLSSFSNQIVGNGYEISKGIIEEYPYSKTTSETSTCESPFSKKQIQIS
INEKPDLNKFVSDVLEPVNALKGDNLPVSVFDPSGVVPLGTTAFEKRGIAISIPIVDMNKCTQCNYCSIVCPHAA
IRPFLLEEVEFEEAPKSMHILRAKGGAEFSSYYYRIQVAPLDCTGCELCVHACPDDALHMEPLQMVRNQEIPHWN
YLVKLPNHGYKFDKSTVKGSQFQKPLLEFSAACEGCGETPYIKLLTQLFGERMVIANATGCSSIWGASFPSVPYT
VTDKGYGPAWGNSLFEDNAEYGLGMVVGYRQRRTRIEALIKEFLNKSDDQKLKNIHEKSAIKDVYLKFEDYLRSW
LKNMNEGDVCQYLYEKITTTIEENLECNKFDTLLSDEHLEMLRRIYQDRDLFPKISHWIVGGDGWAYDIGYAGLD
HVLAYGEDVNILILDTEVYSNTGGQTSKSTPFGAVAKFSQGGNLRQKKDLGLIAMEYGSVYVASIALGANYQQTI
RSLMEAERYPGTSLIIAYSTCIEHGYDKYTLQQESVKLAVESGYWPLYRFNPQLLKFDEINNTIVTLSTGFTLDS
KKIKADISQFLKRENRFLQLFRSNPELASITQSRLKIYSDRRFQHMKNLSENLSVTSLKDQVKKLKDKLLALQNG
EAGGGDLNLQFERNMHILYGTETGNSEDVALYIQAELTSRGYTSTVCNLDDIDIDEFLDPSQYSSFILVTSTAGQ
GEFPGSSKILYESLERRYIELLSNGEDVKFLCNFMQYGVFGLGDSTYVYFNEAAKKWDKLLSDCGAVRIGRMGLG
DDQSDEKYETDLIEWLPDYLQLVNAPEPSNTDDQPKDPLYNVQVIENIYRNDQLNIQTGTLHAINYEGNCDIPIT
PILPPNSILLPLIENTRITSLDHDRDVRHLIFDLSDDSLHKNNLRYNLGDSLALYAQNDFEEAKKACEFFGFNPY
SIIEINLNQIETNKNIRVNQRYLSIFGMKMTILQLFVECLDLWGKPGRRFYHEFYRYCSGSEKEHAKKWSRNEGK
SLIQEFQSETKTFIDMFYLYPSAKPSLSQLLDIVPLIKPRYYSIASSCKYVNNSKIELCVGIVDWNTSSGILKYG
QCTGFINRLPKLISKESNEGIMSDTSNFDIVPVLPCSLKSSAFNLPKDNMSPIIMACMGTGLAPFRAFIQYKYYV
KTVLKQEIGPVILYFGCRYKNKDYLYREELEQYVNDGIITSLNVAFSRDPIEDKKQKLCKDSRIRYRQKVYVQRI
MEENSSELHENLIDKEGYFYLCGTKQVPIDIRKAIVNIIMSQDSNATEESANEILNGLQIKGRYNIEAWS
SEQ ID NO: 12
Synechococcus elongatus PCC 7002 pyruvate:ferredoxin
oxidoreductase (NifJ) amino acid sequence
MATNTIATLDANEAVAKVAYKLNEVIAIYPITPASLMGEWADAWASQGQPNLWGTVPSIIEMQSEGGAAGAVHGA
LQTGSLTTTFTASQGLLLMIPNLYKIAGELTSAVIHVAARSVAAQALSIFGDHSDVMAVRGTGFALLSSASVQEA
HDMALIAQAATMKARVPFIHFFDGFRTSHEIQKIELLDESVLRELIDDEDVFAHRARALTPDHPVVRGTAQNPDV
FFQARESVNPFYDKCSAIVKEMMDRFGALTGRAYKLFEYVGAPDATRVIMLMGSGCETVHETVDYLNAQGEKVGV
LKVRLYRPFDGSALISALPKTVEKIAVLDRTKEPGANGEPLYLDVVSALMEAWEGTMPKVVGGRYGLSSKEFNPA
MVKGIFDELDQAKPKNHFTVGINDDVSHTSLAYDPSFSSEPDSVVRAMFYGLGSDGTVGANKNSIKIIGEETDNY
AQGYFVYDSKKSGAVTVSHLRFGPNLIRSTYLINQANFVGCHQWLFLEKLDVLSGAKDGSIFLLNSPYAVDQVWD
QLPLEVQEQIFHKNLKFYVINANKVARESGMGGRINTVMQTCFFALSGVLPKEEAISKIKEYIQKTYGKKGADVV
TMNIQAVDNTLANLFEVNVGEANSPIRKPPAVSPNAPDFMRNVQAPMLIKEGDRLPVSCLPCDGTYPTGTSKWEK
RNVAQFIPEWDPEVCIQCGKCVMVCPHATIRAKVYEPNLLGNAPESFKSIDAKDKNFSGQKFTIQVAPEDCTGCG
VCVDVCPAKNKAQPSKKAINMVEQLPLREQERTNWDYFLNLPLPERRELKLNQIREQQLQEPLFEFSGACAGCGE
TPYIKLVSQLFGDRTVIANATGCSSIYGGNLPTTPYTTNAEGKGIAWSNSLFEDNAEFGLGFRLSIDKQAQFAAE
LLQRLSGELGDSFVGELLNARQADEADIWEQRQRVRELKNKLATLNSPDAKQLASLADYLVKKSVWIVGGDGWAY
DIGFGGLDHAIASGKNINILVMDTEVYSNTGGQSSKATPRAAVAKFAAGGKPAPKKDLGLIAMTYGNVYVASVAM
GARDEHTLKAFLEAEAYEGPSLIIAYSHCIAHGINMQTAMSHQKELVESGRWLLYRYNPDLKTEGKNPLQLDSRT
PKGSVESSMYKENRFKMLTMTKPKAAKELLKQAQNDVDTRWRMYEYLANRPEA
SEQ ID NO: 13
Zymomonas mobilis pyruvatedecarboxylase (Pdc)
amino acid sequence
MSYTVGTYLAERLVQIGLKHHFAVAGDYNLVLLDNLLLNKNMEQVYCCNELNCGFSAEGYARAKGAAAAVVTYSV
GALSAFDAIGGAYAENLPVILISGAPNNNDHAAGHVLHHALGKTDYHYQLEMAKNITAAAEAIYTPEEAPAKIDH
VIKTALREKKPVYLEIACNIASMPCAAPGPASALFNDEASDEASLNAAVEETLKFIADRDKVAVLVGSKLRAAGA
EEAAVKFADALGGAVATMAAAKSFFPEENPHYIGTSWGEVSYPGVEKTMKEADAVIALAPVFNDYSTTGWTDIPD
PKKLVLAEPRSVVVNGIRFPSVHLKDYLTRLAQKVSKKTGALDFFKSLNAGELKKAAPADPSAPLVNAEIARQVE
ALLTPNTTVIAETGDSWFNAQRIKLPNGARVEYEMQWGHIGWSVPAAFGYAVGAPERRNILMVGDGSFQLTAQEV
AQMVRLKPPVIIFLINNYGYTIEVMIHDGPYNNIKNWDYAGLMEVFNGNGGYDSGAGKGLKAKTGGELAEAIKVA
LANTDGPTLIECFIGREDCTEELVKWGKRVAAANSRKPVNKLL
SEQ ID NO: 14
E. coli NADP-dependent acetaldehyde dehydrogenase
(AldB) amino acid sequence
MTNNPPSAQIKPGEYGFPLKLKARYDNFIGGEWVAPADGEYYQNLTPVTGQLLCEVASSGKRDIDLALDAAHKVK
DKWAHTSVQDRAAILFKIADRMEQNLELLATAETWDNGKPIRETSAADVPLAIDHFRYFASCIRAQEGGISEVDS
ETVAYHFHEPLGVVGQIIPWNFPLLMASWKMAPALAAGNCVVLKPARLTPLSVLLLMEIVGDLLPPGVVNVVNGA
GGVIGEYLATSKRIAKVAFTGSTEVGQQIMQYATQNIIPVTLELGGKSPNIFFADVMDEEDAFFDKALEGFALFA
FNQGEVCTCPSRALVQESIYERFMERAIRRVESIRSGNPLDSVTQMGAQVSHGQLETILNYIDIGKKEGADVLTG
GRRKLLEGELKDGYYLEPTILFGQNNMRVFQEEIFGPVLAVTTFKTMEEALELANDTQYGLGAGVWSRNGNLAYK
MGRGIQAGRVWTNCYHAYPAHAAFGGYKQSGIGRETHKMMLEHYQQTKCLLVSYSDKPLGLF
SEQ ID NO: 15
E. coli acetyl-CoA synthetase (Acs) amino acid sequence
MSQIHKHTIPANIADRCLINPQQYEAMYQQSINVPDTFWGEQGKILDWIKPYQKVKNTSFAPGNVSIKWYEDGTL
NLAANCLDRHLQENGDRTAIIWEGDDASQSKHISYKELHRDVCRFANTLLELGIKKGDVVAIYMPMVPEAAVAML
ACARIGAVHSVIFGGFSPEAVAGRIIDSNSRLVITSDEGVRAGRSIPLKKNVDDALKNPNVTSVEHVVVLKRTGG
KIDWQEGRDLWWHDLVEQASDQHQAEEMNAEDPLFILYTSGSTGKPKGVLHTTGGYLVYAALTFKYVFDYHPGDI
YWCTADVGWVTGHSYLLYGPLACGATTLMFEGVPNWPTPARMAQVVDKHQVNILYTAPTAIRALMAEGDKAIEGT
DRSSLRILGSVGEPINPEAWEWYWKKIGNEKCPVVDTWWQTETGGFMITPLPGATELKAGSATRPFFGVQPALVD
NEGNPLEGATEGSLVITDSWPGQARTLFGDHERFEQTYFSTFKNMYFSGDGARRDEDGYYWITGRVDDVLNVSGH
RLGTAEIESALVAHPKIAEAAVVGIPHNIKGQAIYAYVTLNHGEEPSPELYAEVRNWVRKEIGPLATPDVLHWTD
SLPKTRSGKIMRRILRKIAAGDTSNLGDTSTLADPGVVEKLLEEKQAIAMPS

What is claimed is:

1. An engineered photosynthetic microbe, wherein said engineered photosynthetic microbe comprises a recombinant MdhP enzyme.

2. The engineered photosynthetic microbe of claim 1, wherein said recombinant MdhP enzyme is a Pisum sativum MdhP enzyme.

3. The engineered photosynthetic microbe of claim 1, wherein said recombinant MdhP enzyme is at least 95% identical to SEQ ID NO: 1.

4. The engineered photosynthetic microbe of claim 1, wherein said recombinant MdhP enzyme is at least 95% identical to SEQ ID NO: 2.

5. The engineered photosynthetic microbe of claim 1, wherein said engineered photosynthetic microbe comprises an additional mutation which reduces the expression or activity of its endogenous Mdh enzyme.

6. The engineered photosynthetic microbe of claim 5, wherein said mutation is a knockout of the gene encoding said endogenous Mdh enzyme.

7. The engineered photosynthetic microbe of claim 1, wherein said engineered photosynthetic microbe further comprises a recombinant phosphoenol pyruvate carboxylase.

8. The engineered photosynthetic microbe of claim 1, wherein said engineered photosynthetic microbe further comprises a recombinant NADPH-linked malic enzyme.

9. The engineered photosynthetic microbe of claim 1, wherein said engineered photosynthetic microbe further comprises a recombinant phosphoenol pyruvate carboxylase and a recombinant NADPH-linked malic enzyme.

10. The engineered photosynthetic microbe of claim 7 or 9, wherein said recombinant phosphoenol pyruvate carboxylase is the S8D mutant phosphoenol pyruvate carboxylase.

11. The engineered photosynthetic microbe of claim 10, wherein said S8D mutant phosphoenol pyruvate carboxylase is derived from Sorghum bicolor Ppc.

12. The engineered photosynthetic microbe of claim 10, wherein said recombinant phosphoenol pyruvate carboxylase is at least 95% identical to SEQ ID NO: 4.

13. The engineered photosynthetic microbe of claim 8 or 9, wherein said recombinant NADPH-linked malic enzyme is the Synechococcus elongatus PPC 7002 NADPH-linked malic enzyme.

14. The engineered photosynthetic microbe of claim 8 or 9, wherein said recombinant NADPH-linked malic enzyme is at least 95% identical to SEQ ID NO: 5.

15. An engineered photosynthetic microbe, wherein said engineered photosynthetic microbe comprises a recombinant oxaloacetate decarboxylase.

16. The engineered photosynthetic microbe of claim 15, wherein said recombinant oxaloacetate decarboxylase is Corynebacterium glutamicum oxaloacetate decarboxylase.

17. The engineered photosynthetic microbe of claim 15, wherein said recombinant oxaloacetate decarboxylase is at least 95% identical to SEQ ID NO: 6.

18. The engineered photosynthetic microbe of claim 15, wherein said engineered photosynthetic microbe further comprises a recombinant phosphoenol pyruvate carboxylase.

19. The engineered photosynthetic microbe of claim 18, wherein said recombinant phosphoenol pyruvate carboxylase is at least 95% identical to SEQ ID NO: 4.

20. The engineered photosynthetic microbe of claim 15, wherein said engineered photosynthetic microbe comprises an endogenous, non-recombinant phosphoenol pyruvate carboxylase.

21. The engineered photosynthetic microbe of claim 15, wherein said engineered photosynthetic microbe further comprises a recombinant phosphoenolpyruvate carboxykinase.

22. The engineered photosynthetic microbe of claim 21, wherein said recombinant phosphoenolpyruvate carboxykinase is derived from E. Coli.

23. The engineered photosynthetic microbe of claim 21, wherein said recombinant phosphoenolpyruvate carboxykinase is at least 95% identical to SEQ ID NO: 7.

24. The engineered photosynthetic microbe of claim 15, wherein said engineered photosynthetic microbe lacks an endogenous or recombinant malate dehydrogenase activity, or wherein said engineered photosynthetic microbe comprises a mutation which attenuates or knocks out endogenous malate dehydrogenase activity in said engineered photosynthetic microbe.

25. The engineered photosynthetic microbe of any of claims 1-24, wherein said engineered photosynthetic microbe further comprises a mutation which attenuates or knocks out endogenous pyruvate dehydrogenase activity in said photosynthetic microbe.

26. An engineered photosynthetic microbe, wherein said engineered photosynthetic microbe comprises a recombinant NADPH-producing transhydrogenase system.

27. The engineered photosynthetic microbe of claim 26, further comprising a recombinant MdhP enzyme.

28. The engineered photosynthetic microbe of claim 26, wherein said recombinant NADPH-producing transhydrogenase system comprises PntA transhydrogenase, PntB transhydrogenase, or PntAB transhydrogenase.

29. The engineered photosynthetic microbe of claim 28, wherein said PntA transhydrogenase comprises a sequence at least 95% identical to SEQ ID NO: 8.

30. The engineered photosynthetic microbe of claim 28, wherein said PntB transhydrogenase comprises a sequence at least 95% identical to SEQ ID NO: 9.

31. The engineered photosynthetic microbe of claim 27, wherein said PntAB transhydrogenase comprises a sequence at least 95% identical to SEQ ID NO: 8 and further comprises a sequence at least 95% identical to SEQ ID NO: 9.

32. An engineered photosynthetic microbe, wherein said engineered photosynthetic microbe comprises a recombinant NADPH-generating pyruvate dehydrogenase.

33. The engineered photosynthetic microbe of claim 32, further comprising a recombinant MdhP enzyme.

34. The engineered photosynthetic microbe of claim 32, wherein said recombinant NADPH-generating pyruvate dehydrogenase is Euglena gracilis Pno or Cryptosporidium parvum Pno.

35. The engineered photosynthetic microbe of claim 34, wherein said recombinant NADPH-generating pyruvate dehydrogenase is at least 95% identical to SEQ ID NO: 10.

36. The engineered photosynthetic microbe of claim 34, wherein said recombinant NADPH-generating pyruvate dehydrogenase is at least 95% identical to SEQ ID NO: 11.

37. The engineered photosynthetic microbe of claim 32, wherein said engineered photosynthetic microbe naturally lacks an endogenous pyruvate dehydrogenase activity or comprises a mutation which attenuates or knocks out endogenous pyruvate dehydrogenase activity.

38. An engineered photosynthetic microbe, wherein said engineered photosynthetic microbe comprises a recombinant pyruvate:ferredoxin oxidoreductase, wherein expression of said recombinant pyruvate:ferredoxin oxidoreductase is expressed by a gene, wherein said gene is controlled by a promoter which leads to increased expression of said pyruvate:ferredoxin oxidoreductase relative to that obtained with the endogenous gene under the control of its native promoter, or wherein said gene is present in a copy number which leads to increased expression of said pyruvate:ferredoxin oxidoreductase relative to that obtained with an otherwise identical photosynthetic microbe with a lower copy number.

39. The engineered photosynthetic microbe of claim 38, further comprising a recombinant MdhP enzyme.

40. The engineered photosynthetic microbe of claim 38, wherein said recombinant pyruvate:ferredoxin oxidoreductase is at least 95% identical to SEQ ID NO: 12.

41. An engineered photosynthetic microbe, wherein said engineered photosynthetic microbe comprises a recombinant NADPH-generating pyruvate dehydrogenase system, wherein said recombinant NADPH-generating pyruvate dehydrogenase system comprises a pyruvate decarboxylase, an NADP-dependent acetaldehyde dehydrogenase, and an acetyl-CoA synthetase.

42. The engineered photosynthetic microbe of claim 41, further comprising a recombinant MdhP enzyme

43. The engineered photosynthetic microbe of claim 41, wherein said pyruvate decarboxylase is Zymomonas mobilis pyruvate decarboxylase.

44. The engineered photosynthetic microbe of claim 41, wherein said pyruvate decarboxylase is at least 95% identical to SEQ ID NO: 13.

45. The engineered photosynthetic microbe of claim 41, wherein said NADP-dependent acetaldehyde dehydrogenase is E. coli AldB.

46. The engineered photosynthetic microbe of claim 41, wherein said NADP-dependent acetaldehyde dehydrogenase is at least 95% identical to SEQ ID NO: 14.

47. The engineered photosynthetic microbe of claim 41, wherein said acetyl-CoA synthetase is E. coli Acs.

48. The engineered photosynthetic microbe of claim 41, wherein said acetyl-CoA synthetase is at least 95% identical to SEQ ID NO: 15.

49. The engineered photosynthetic microbe of any of claims 1-48, wherein said engineered photosynthetic microbe further comprises at least one recombinant gene selected from the group consisting of pyruvate decarboxylase and alcohol dehydrogenase.

50. A method for improving production of a carbon-based compound of interest by a photosynthetic microbe, wherein said carbon-based compound of interest is synthesized by said photosynthetic microbe using pyruvate, at least in part, as a source of carbon, comprising: (a) culturing said photosynthetic microbe in the presence of light and an inorganic carbon source, and (b) recombinantly expressing an MdhP enzyme in said photosynthetic microbe.

51. The method of claim 50, wherein said recombinant expression of said MdhP enzyme in said photosynthetic microbe results in increased carbon flux to pyruvate in said photosynthetic microbe.

52. The method of claim 50, wherein said MdhP enzyme is a Pisum sativum MdhP enzyme.

53. The method of claim 50, wherein said MdhP enzyme is at least 95% identical to SEQ ID NO: 1.

54. The method of claim 50, wherein said MdhP enzyme is at least 95% identical to SEQ ID NO: 2.

55. The method of claim 50, wherein said photosynthetic microbe comprises an additional mutation which reduces the expression or activity of its endogenous Mdh enzyme.

56. The method of claim 54, wherein said mutation is a knockout of the gene encoding said endogenous Mdh enzyme.

57. The method of claim 50, wherein said method further comprises recombinantly expressing a phosphoenolpyruvate carboxylase enzyme.

58. The method of claim 50, wherein said method further comprises recombinantly expressing a recombinant NADPH-linked malic enzyme.

59. The method of claim 50, wherein said method further comprises recombinantly expressing a phosphoenolpyruvate carboxylase enzyme and an NADPH-linked malic enzyme.

60. The method of any of claims 50-59, wherein said recombinant expression results in increased carbon flux to pyruvate in said photosynthetic microbe.

61. The method of claim 57 or 59, wherein said recombinant phosphoenol pyruvate carboxylase is the S8D mutant phosphoenol pyruvate carboxylase.

62. The method of claim 61, wherein said S8D mutant phosphoenol pyruvate carboxylase is derived from Sorghum ppc.

63. The method of claim 61, wherein said S8D mutant phosphoenol pyruvate carboxylase is at least 95% identical to SEQ ID NO: 4.

64. The method of claim 58 or 59, wherein said recombinant NADPH-linked malic enzyme is the Synechococcus elongatus PPC 7002 NADPH-linked malic enzyme.

65. The method of claim 58 or 59, wherein said recombinant NADPH-linked malic enzyme is at least 95% identical to SEQ ID NO: 5.

66. A method for improving production of a carbon-based compound of interest by a photosynthetic microbe, wherein said carbon-based compound of interest is synthesized by said photosynthetic microbe using pyruvate, at least in part, as a source of carbon, comprising: (a) culturing said photosynthetic microbe in the presence of light and an inorganic carbon source, and (b) recombinantly expressing an oxaloacetate decarboxylase enzyme in said photosynthetic microbe.

67. The method of claim 66, wherein said recombinant expression of said oxaloacetate decarboxylase enzyme in said photosynthetic microbe results in increased carbon flux to pyruvate in said photosynthetic microbe.

68. The method of claim 66, wherein said oxaloacetate decarboxylase is Corynebacterium glutamicum oxaloacetate decarboxylase.

69. The method of claim 66, wherein said oxaloacetate decarboxylase is at least 95% identical to SEQ ID NO: 6.

70. The method of claim 66, wherein said photosynthetic microbe further comprises a recombinant phosphoenol pyruvate carboxylase.

71. The method of claim 70, wherein said recombinant phosphoenol pyruvate carboxylase is at least 95% identical to SEQ ID NO: 4.

72. The method of claim 66, wherein said photosynthetic microbe comprises an endogenous, non-recombinant phosphoenol pyruvate carboxylase.

73. The method of claim 66, wherein said method further comprises recombinantly expressing a phosphoenolpyruvate carboxykinase in said photosynthetic microbe.

74. The method of claim 73, wherein said phosphoenolpyruvate carboxykinase is derived from E. Coli.

75. The method of claim 73, wherein said phosphoenolpyruvate carboxykinase is at least 95% identical to SEQ ID NO: 7.

76. The method of claim 66, wherein said photosynthetic microbe lacks an endogenous or recombinant malate dehydrogenase activity, or wherein said engineered photosynthetic microbe comprises a mutation which attenuates or knocks out endogenous malate dehydrogenase activity in said engineered photosynthetic microbe.

77. The method of any of claims 50-76, wherein said engineered photosynthetic microbe further comprises a mutation which attenuates or knocks out endogenous pyruvate dehydrogenase activity in said photosynthetic microbe.

78. A method for improving production of a carbon-based compound of interest by a photosynthetic microbe, wherein said carbon-based compound of interest is synthesized by said photosynthetic microbe using acetyl-CoA, at least in part, as a source of carbon, comprising: (a) culturing said photosynthetic microbe in the presence of light and an inorganic carbon source, and (b) recombinantly expressing an NADPH-producing transhydrogenase system in said photosynthetic microbe.

79. The method of claim 78, wherein said method further comprises recombinantly expressing an MdhP enzyme in said photosynthetic microbe.

80. The method of claim 78, wherein said recombinant expression of said NADPH-producing transhydrogenase in said photosynthetic microbe results in increased carbon flux to acetyl-CoA in said photosynthetic microbe.

81. The method of claim 78, wherein said NADPH-producing transhydrogenase system comprises PntA transhydrogenase, PntB transhydrogenase, or PntAB transhydrogenase.

82. The method of claim 81, wherein said PntA transhydrogenase is at least 95% identical to SEQ ID NO: 8.

83. The method of claim 81, wherein said PntB transhydrogenase is at least 95% identical to SEQ ID NO: 9.

84. The method of claim 81, wherein said PntAB transhydrogenase is at least 95% identical to SEQ ID NO: 8 and further comprises a sequence at least 95% identical to SEQ ID NO: 9.

85. A method for improving production of a carbon-based compound of interest by a photosynthetic microbe, wherein said carbon-based compound of interest is synthesized by said photosynthetic microbe using acetyl-CoA, at least in part, as a source of carbon, comprising: (a) culturing said photosynthetic microbe in the presence of light and an inorganic carbon source, and (b) recombinantly expressing an NADPH-generating pyruvate dehydrogenase in said photosynthetic microbe.

86. The method of claim 85, wherein said method further comprises recombinantly expressing an MdhP enzyme in said photosynthetic microbe.

87. The method of claim 85, wherein said recombinant expression of said NADPH-generating pyruvate dehydrogenase in said photosynthetic microbe results in increased carbon flux to acetyl-CoA in said photosynthetic microbe.

88. The method of claim 85, wherein said NADPH-generating pyruvate dehydrogenase is Euglena gracilis Pno or Cryptosporidium parvum Pno.

89. The method of claim 85, wherein said NADPH-generating pyruvate dehydrogenase is at least 95% identical to SEQ ID NO: 10.

90. The method of claim 85, wherein said NADPH-generating pyruvate dehydrogenase is at least 95% identical to SEQ ID NO: 11.

91. The method of claim 85, wherein said photosynthetic microbe naturally lacks an endogenous pyruvate dehydrogenase activity or comprises a mutation which attenuates or knocks out endogenous pyruvate dehydrogenase activity.

92. A method for improving production of a carbon-based compound of interest by a photosynthetic microbe, wherein said carbon-based compound of interest is synthesized by said photosynthetic microbe using acetyl-CoA, at least in part, as a source of carbon, comprising: (a) culturing said photosynthetic microbe in the presence of light and an inorganic carbon source, and (b) recombinantly expressing a pyruvate:ferredoxin oxidoreductase in said photosynthetic microbe, wherein expression of said recombinant pyruvate:ferredoxin oxidoreductase is expressed by a gene, wherein said gene is controlled by a promoter which leads to increased expression of said pyruvate:ferredoxin oxidoreductase relative to that obtained with the endogenous gene under the control of its native promoter, or wherein said gene is present in a copy number which leads to increased expression of said pyruvate:ferredoxin oxidoreductase relative to that obtained with an otherwise identical photosynthetic microbe with a lower copy number.

93. The method of claim 92, wherein said method further comprises recombinantly expressing an MdhP enzyme in said photosynthetic microbe.

94. The method of claim 92, wherein said recombinant expression of said pyruvate:ferredoxin oxidoreductase in said photosynthetic microbe results in increased carbon flux to acetyl-CoA in said photosynthetic microbe

95. The method of claim 92, wherein said pyruvate:ferredoxin oxidoreductase is at least 95% identical to SEQ ID NO: 12.

96. A method for improving production of a carbon-based compound of interest by a photosynthetic microbe, wherein said carbon-based compound of interest is synthesized by said photosynthetic microbe using acetyl-CoA, at least in part, as a source of carbon, comprising: (a) culturing said photosynthetic microbe in the presence of light and an inorganic carbon source, and (b) recombinantly expressing an NADPH-generating pyruvate dehydrogenase system in said photosynthetic microbe, wherein said NADPH-generating pyruvate dehydrogenase system comprises a pyruvate decarboxylase, an NADP-dependent acetaldehyde dehydrogenase, and an acetyl-CoA synthetases.

97. The method of claim 96, wherein said method further comprises recombinantly expressing an MdhP enzyme in said photosynthetic microbe.

98. The method of claim 96, wherein said recombinant expression of said NADPH-generating pyruvate dehydrogenase system in said photosynthetic microbe results in increased carbon flux to acetyl-CoA in said photosynthetic microbe.

99. The method of claim 96, wherein said pyruvate decarboxylase is Zymomonas mobilis pyruvate decarboxylase.

100. The method of claim 96, wherein said pyruvate decarboxylase is at least 95% identical to SEQ ID NO: 13.

101. The method of claim 96, wherein said NADP-dependent acetaldehyde dehydrogenase is E. coli AldB.

102. The method of claim 96, wherein said NADP-dependent acetaldehyde dehydrogenase is at least 95% identical to SEQ ID NO: 14.

103. The method of claim 96, wherein said acetyl-CoA synthetase is E. coli Acs.

104. The method of claim 96, wherein said acetyl-CoA synthetase is at least 95% identical to SEQ ID NO: 15.

105. The method of any of claims 50-104, wherein said carbon-based compound of interest is produced at a greater rate or in greater yields in said engineered photosynthetic microbe relative to an otherwise identical photosynthetic microbe lacking the recited recombinant enzymes or mutations.

106. The method of claim 105, wherein said engineered photosynthetic microbe further comprises at least one recombinant gene selected from the group consisting of pyruvate decarboxylase and alcohol dehydrogenase.

107. The method of claim 106, wherein said carbon-based compound of interest is ethanol.

108. The method of any of claims 50-104, wherein said carbon-based compound of interest is selected from the group consisting of: alcohols, alkenes, and alkanes.

109. An engineered heterotrophic microbe, wherein said engineered heterotrophic microbe comprises a recombinant MdhP enzyme.

110. An engineered heterotrophic microbe, wherein said engineered heterotrophic microbe comprises a recombinant oxaloacetate decarboxylase.

111. An engineered heterotrophic microbe, wherein said engineered heterotrophic microbe comprises a recombinant NADPH-producing transhydrogenase system.

112. An engineered heterotrophic microbe, wherein said engineered heterotrophic microbe comprises a recombinant NADPH-generating pyruvate dehydrogenase.

113. An engineered heterotrophic microbe, wherein said engineered heterotrophic microbe comprises a recombinant pyruvate:ferredoxin oxidoreductase, wherein expression of said recombinant pyruvate:ferredoxin oxidoreductase is expressed by a gene, wherein said gene is controlled by a promoter which leads to increased expression of said pyruvate:ferredoxin oxidoreductase relative to that obtained with the endogenous gene under the control of its native promoter, or wherein said gene is present in a copy number which leads to increased expression of said pyruvate:ferredoxin oxidoreductase relative to that obtained with an otherwise identical heterotrophic microbe with a lower copy number.

114. An engineered heterotrophic microbe, wherein said engineered heterotrophic microbe comprises a recombinant NADPH-generating pyruvate dehydrogenase system, wherein said recombinant NADPH-generating pyruvate dehydrogenase system comprises a pyruvate decarboxylase, an NADP-dependent acetaldehyde dehydrogenase, and an acetyl-CoA synthetase.

115. A method for improving production of a carbon-based compound of interest by a heterotrophic microbe, wherein said carbon-based compound of interest is synthesized by said heterotrophic microbe using pyruvate, at least in part, as a source of carbon, comprising: (a) culturing said heterotrophic microbe in the presence of light and an inorganic carbon source, and (b) recombinantly expressing an MdhP enzyme in said heterotrophic microbe.

116. A method for improving production of a carbon-based compound of interest by a heterotrophic microbe, wherein said carbon-based compound of interest is synthesized by said heterotrophic microbe using pyruvate, at least in part, as a source of carbon, comprising: (a) culturing said heterotrophic microbe in the presence of light and an inorganic carbon source, and (b) recombinantly expressing an oxaloacetate decarboxylase enzyme in said heterotrophic microbe.

117. A method for improving production of a carbon-based compound of interest by a heterotrophic microbe, wherein said carbon-based compound of interest is synthesized by said heterotrophic microbe using acetyl-CoA, at least in part, as a source of carbon, comprising: (a) culturing said heterotrophic microbe in the presence of light and an inorganic carbon source, and (b) recombinantly expressing an NADPH-producing transhydrogenase system in said heterotrophic microbe.

118. A method for improving production of a carbon-based compound of interest by a heterotrophic microbe, wherein said carbon-based compound of interest is synthesized by said heterotrophic microbe using acetyl-CoA, at least in part, as a source of carbon, comprising: (a) culturing said heterotrophic microbe in the presence of light and an inorganic carbon source, and (b) recombinantly expressing an NADPH-generating pyruvate dehydrogenase in said heterotrophic microbe.

119. A method for improving production of a carbon-based compound of interest by a heterotrophic microbe, wherein said carbon-based compound of interest is synthesized by said heterotrophic microbe using acetyl-CoA, at least in part, as a source of carbon, comprising: (a) culturing said heterotrophic microbe in the presence of light and an inorganic carbon source, and (b) recombinantly expressing a pyruvate:ferredoxin oxidoreductase in said heterotrophic microbe, wherein expression of said recombinant pyruvate:ferredoxin oxidoreductase is expressed by a gene, wherein said gene is controlled by a promoter which leads to increased expression of said pyruvate:ferredoxin oxidoreductase relative to that obtained with the endogenous gene under the control of its native promoter, or wherein said gene is present in a copy number which leads to increased expression of said pyruvate:ferredoxin oxidoreductase relative to that obtained with an otherwise identical heterotrophic microbe with a lower copy number.

120. A method for improving production of a carbon-based compound of interest by a heterotrophic microbe, wherein said carbon-based compound of interest is synthesized by said heterotrophic microbe using acetyl-CoA, at least in part, as a source of carbon, comprising: (a) culturing said heterotrophic microbe in the presence of light and an inorganic carbon source, and (b) recombinantly expressing an NADPH-generating pyruvate dehydrogenase system in said heterotrophic microbe, wherein said NADPH-generating pyruvate dehydrogenase system comprises a pyruvate decarboxylase, an NADP-dependent acetaldehyde dehydrogenase, and an acetyl-CoA synthetases.
